(19) United States
(12) Patent Application Publication
(10) Pub. No.: US 2026/0139234 A1
(43) Pub. Date: May 21, 2026

(54) METHOD FOR THE PRODUCTION OF RECOMBINANT AAV PARTICLE PREPARATIONS

(71) Applicant: Hoffmann-La Roche Inc., Little Falls, NJ (US)

(72) Inventors: Julia EMMENECKER, Eurasburg (DE); Sven MARKERT, Sindelsdorf (DE)

(73) Assignee: Hoffmann-La Roche Inc., Little Falls, NJ (US)

(21) Appl. No.: 19/331,653

(22) Filed: Sep. 17, 2025

Related U.S. Application Data
(63) Continuation of application No. PCT/EP2024/057207, filed on Mar. 19, 2024.

(30) Foreign Application Priority Data
Mar. 21, 2023 (EP) 23163162.3

(51) Int. Cl. C12N 7/02; A61K 35/76

(52) U.S. Cl. CPC C12N 7/025; A61K 35/76; C12N 2750/14122; C12N 2750/14132; C12N 2750/14152

(57) ABSTRACT

Herein is reported a method for producing recombinant adeno-associated viral particle preparation (rAAVp) comprising the step of cultivating a mammalian cell comprising expression cassettes for a non-adeno-associated viral gene, which is interspaced between two AAV inverted terminal repeats (ITRs), an adeno-associated virus rep gene, an adeno-associated virus cap gene, an adeno-associated virus E1A gene, an adeno-associated virus E1B gene, an adeno-associated virus E2A gene, an adeno-associated virus E4orf6 and an adeno-associated virus VA RNA gene, and thereby producing the rAAVp, wherein the cultivating is at a pH value in the range of and including pH 7.4 to pH 7.6. The yield of the rAAVp produced by the cultivating at a pH value in the range of and including pH 7.4 to pH 7.6 is higher than the yield of a rAAVp produced by a cultivating at a pH value in the range of and including pH 7.0 to pH 7.2 and the rAAVp produced by the cultivating at a pH value in the range of and including pH 7.4 to pH 7.6 has a higher percentage of full particles than a rAAVp produced by a cultivating at a pH value in the range of and including pH 7.0 to pH 7.2.

The current invention is in the field of gene therapy. More specifically, the current invention is directed to a method for producing recombinant adeno-associated viral particles comprising a therapeutic transgene in mammalian cells, especially CHO and HEK cells, at a pH value that is higher as generally used in the art.

### BACKGROUND

Gene therapy is opening unprecedented opportunities for novel therapeutic approaches. Based on the concept of rescuing function mutations by co-expressing the correct gene, to allow biological functions to be restored, it requires the use of viral vectors to ensure the proper delivery of therapeutic genes. In this context, recombinant adeno-associated viruses (rAAV) are the most widely used vectors.

Bioprocessing of viruses is complex and requires systematic and coordinated steps both in upstream processing as well as downstream processing. However, the use of traditional cultivation processes for the production of therapeutic viruses does not support effective commercial manufacturing strategies. This is even more pronounced by the large size of viruses as compared to therapeutic biomolecules, such as, e.g., antibodies. Additionally, viruses are much more complex.

The bio manufacturing process of therapeutic viruses requires the insertion of the therapeutic transgene into the recombinant AAV capsid shell (full r AAV particles, i.e. recombinant AAV particles comprising an encapsidated nucleic acid). However, a percentage of rAAV that do not contain the desired transgene (empty recombinant AAV particles, i.e. rAAV particles not comprising an encapsidated nucleic acid) might also be produced, as well as partly filled rAAVs (partly filled recombinant AAV particles).

### SUMMARY OF EMBODIMENTS OF THE INVENTION

The current invention is based, at least in part, on the finding that the productivity of mammalian cells producing a recombinant adeno-associated viral particle can be increased when the cultivation is carried out at elevated pH values, such as pH 7.4-7.6.

The current invention is based, at least in part, on the finding that the fraction of full recombinant AAV particles obtained from a cultivation of mammalian cells producing said recombinant adeno-associated viral particle can be increased when the cultivation is carried out at elevated pH values, such as pH 7.4-7.6.

The current invention comprises at least the following embodiments:
1. A method for producing a recombinant adeno-associated viral particle preparation (rAAVp) comprising the step of cultivating a mammalian cell comprising expression cassettes for a non-adeno-associated viral gene, which is interspaced between two AAV inverted terminal repeats (ITRs), for an adeno-associated virus rep gene, for an adeno-associated virus cap gene, for an adeno-associated virus E1A gene, for an adeno-associated virus E1B gene, for an adeno-associated virus E2A gene, for an adeno-associated virus E4orf6 and optionally for an adeno-associated virus VA RNA gene, and thereby producing the rAAVp,
   wherein the cultivating is at a pH value in the range of and including pH 7.4 to pH 7.6.
2. A method for producing a recombinant adeno-associated viral particle preparation (rAAVp) comprising the step of cultivating a HEK cell comprising expression cassettes for a non-adeno-associated viral gene, which is interspaced between two AAV inverted terminal repeats (ITRs), for an adeno-associated virus rep gene, for an adeno-associated virus cap gene, for an adeno-associated virus E2A gene, for an adeno-associated virus E4orf6 and optionally for an adeno-associated virus VA RNA gene, and thereby producing the rAAVp,
   wherein the cultivating is at a pH value in the range of and including pH 7.4 to pH 7.6.
3. The method according to any one of embodiments 1 or 2, wherein the mammalian cell is a HEK293 cell.
4. The method according to any one of embodiments 1 to 3, wherein the genomic titer of the rAAVp produced by the cultivating at a pH value in the range of and including pH 7.4 to pH 7.6 is higher than the capsid titer of a rAAVp produced by a cultivating at a pH value in the range of and including pH 7.0 to pH 7.2.
5. The method according to embodiment 4, wherein the genomic titer is at least 1.5 times higher.
6. The method according to any one of embodiments 4 to 5, wherein the genomic titer is at least 2 times higher.
7. The method according to any one of embodiments 4 to 6, wherein the genomic titer is at least 6 times higher.
8. The method according to any one of embodiments 4 to 7, wherein the genomic titer is at least 10 times higher.
9. The method according to any one of embodiments 1 to 8, wherein the capsid titer of the rAAVp produced by the cultivating at a pH value in the range of and including pH 7.4 to pH 7.6 is higher than the capsid titer of a rAAVp produced by a cultivating at a pH value in the range of and including pH 7.0 to pH 7.2.
10. The method according to embodiment 9, wherein the capsid titer is at least 1.5 times higher.
11. The method according to any one of embodiments 9 to 10, wherein the capsid titer is at least 2 times higher.
12. The method according to any one of embodiments 9 to 11, wherein the capsid titer is at least 3 times higher.
13. The method according to any one of embodiments 1 to 12, wherein the genomic titer and the capsid titer of the rAAVp produced by the cultivating at a pH value in the range of and including pH 7.4 to pH 7.6 is higher than the genomic titer and the capsid titer of a rAAVp produced by a cultivating at a pH value in the range of and including pH 7.0 to pH 7.2.
14. The method according to embodiment 13, wherein the genomic titer and the capsid titer is at least 1.5 times higher.
15. The method according to any one of embodiments 13 to 14, wherein the genomic titer and the capsid titer is at least 2 times higher.
16. The method according to any one of embodiments 13 to 15, wherein the genomic titer and the capsid titer is at least 3 times higher.
17. The method according to any one of embodiments 13 to 16, wherein the genomic titer is at least 4 times higher and the capsid titer is at least 2 times higher.
18. The method according to any one of embodiments 13 to 17, wherein the genomic titer is at least 6 times higher and the capsid titer is at least 2 times higher.
19. The method according to any one of embodiments 1 to 20, wherein the rAAVp produced by the cultivating at a pH value in the range of and including pH 7.4 to pH 7.6 has a higher percentage of full particles than a rAAVp produced by a cultivating at a pH value in the range of and including pH 7.0 to pH 7.2.
20. The method according to embodiment 19, wherein the percentage of full particles is at least 1.5 times higher.
21. The method according to any one of embodiments 19 to 20, wherein the percentage of full particles is at least 2 times higher.
22. The method according to any one of embodiments 19 to 21, wherein the percentage of full particles is at least 4 times higher.
23. The method according to any one of embodiments 1 to 22, wherein the rAAVp is a therapeutic rAAVp.
24. The method according to any one of embodiments 1 to 23, wherein the rAAVp is for transfer of a nucleic acid that is transcribed into a polypeptide with therapeutic effect into target cells.
25. The method according to any one of embodiments 1 to 24, wherein the rAAVp is for transfer of a nucleic acid that has therapeutic effect into target cells.
26. The method according to any one of embodiments 1 to 25, wherein the rAAVp comprises recombinant adeno-associated viral particles (rAAVs) comprising at least one coding nucleic acid sequence interspaced between two adeno-associated viral inverted terminal repeats.
27. The method according to any one of embodiments 1 to 26, wherein the rAAV in the rAAVp is derived from a wild-type AAV particle selected from the group consisting of AAV1, AAV2, AAV3, AAV4, AAV5, AAV6, AAV7, AAV8, AAV9, AAV10, AAV11, AAV12, AAV 2i8, AAV rh.74, AAV rh.10 and AAV 7m8, as well as variants thereof.
28. The method according to any one of embodiments 1 to 27, wherein the rAAV is of the serotype AAV2 or a variant thereof.
29. The method according to any one of embodiments 1 to 28, wherein the rAAV comprises one or two ITR sequences of a wild-type AAV1, AAV2, AAV3, AAV4, AAV5, AAV6, AAV7, AAV8, AAV9, AAV10, AAV 11 or AAV12.
30. The method according to any one of embodiments 1 to 29, wherein the cultivating encompasses the inoculation of the bioreactor and the harvest of the rAAVp.
31. The method according to any one of embodiments 1 to 30, wherein the cultivating starts with the inoculation of the bioreactor.
32. The method according to any one of embodiments 1 to 31, wherein one or more or all of the expression cassettes for the non-adeno-associated viral gene, which is interspaced between two AAV ITRs, for the adeno-associated virus rep gene, for the adeno-associated virus cap gene, for the adeno-associated virus E2A gene, for the adeno-associated virus E4orf6 and for the adeno-associated virus VA RNA gene are introduced into the mammalian cell or HEK cell after the inoculation of the bioreactor.
33. The method according to any one of embodiments 1 to 32, wherein one or more or all of the expression cassettes for the non-adeno-associated viral gene, which is interspaced between two AAV ITRs, for the adeno-associated virus rep gene, for the adeno-associated virus cap gene, for the adeno-associated virus E2A gene, for the adeno-associated virus E4orf6 and optionally for the adeno-associated virus VA RNA gene are introduced into the mammalian cell or the HEK cell after the inoculation of the bioreactor, whereby up to three plasmids are co-transfected into the mammalian cell, whereby one of the plasmids comprises the expression cassette for the non-adeno-associated viral gene, which is interspaced between two AAV ITRs, one of the plasmids comprises the expression cassettes for the rep and cap genes and one of the plasmids comprises the expression cassettes for the adenoviral E2A, E4orf6 and VA RNA genes.
34. The method according to any one of embodiments 1 to 33, wherein expression of one or more or all of the non-adeno-associated viral gene, which is interspaced between two AAV ITRs, the adeno-associated virus rep gene, the adeno-associated virus cap gene, the adeno-associated virus E2A gene, the adeno-associated virus E4orf6 and optionally the adeno-associated virus VA RNA gene is induced after the inoculation of the bioreactor.
35. The method according to any one of embodiments 32 to 33, wherein the introduction is about 16 to 32 hours after the inoculation of the bioreactor.
36. The method according to embodiment 34, wherein the induction is about 16 to 32 hours after the inoculation of the bioreactor.
37. The method according to any one of embodiments 35 to 36, wherein the introduction or induction is about 24 hours after the inoculation of the bioreactor.
38. The method according to any one of embodiments 1 to 37, wherein the method further comprises after the cultivating step the step of isolating the rAAV from the cells and/or the cultivation medium and optionally purifying the rAAV.
39. The method according to embodiment 38, wherein the purifying is by one or more column chromatography steps and/or a CsCl or iodixanol gradient centrifugation step.
40. The method according to embodiment 38 or 39, wherein the first chromatography step is an affinity chromatography step.
41. The method according to any one of embodiments 38 to 40, wherein the purifying is by a sequence of chromatography steps wherein the first is an affinity chromatography, followed by an anion exchange chromatography or a cation exchange chromatography, and an optional size exclusion chromatography.
42. A pharmaceutical composition comprising the rAAVp obtained with a method according to any one of embodiments 1 to 41.
43. A pharmaceutical composition comprising the rAAVp obtained with a method according to any one of embodiments 1 to 41 and a pharmaceutically acceptable excipient.
44. Use of the method according to any one of embodiments 1 to 41 for increasing the yield of a recombinantly produced rAAVp.
45. Use of the method according to any one of embodiments 1 to 41 for increasing the percentage of full particles in a rAAVp.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### BRIEF DESCRIPTION OF THE DRAWINGS

FIG. 1 Visualization of the genomic titer in the harvested samples of Set 1 (harvest 120 hours post transfection, no lysis).

FIG. 2 Visualization of the capsid titer in the harvested samples of Set 1 (harvest 120 hours post transfection, no lysis).

FIG. 3 Visualization of the full-to-empty ratio in the harvested samples of Set 1 (harvest 120 hours post transfection, no lysis).

### DETAILED DESCRIPTION

The current invention is based, at least in part, on the finding that the productivity of mammalian cells producing a recombinant adeno-associated viral particle can be increased when the cultivation is carried out at elevated pH values, such as pH 7.4-7.6.

### Definitions

Unless otherwise defined herein, scientific and technical terms used in connection with the current invention shall have the meanings that are commonly understood by those of ordinary skill in the art. Further, unless otherwise required by context, singular terms shall include pluralities and plural terms shall include the singular.

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R.I., Culture of Animal Cells: A Manual of Basic Technique, second edition, Alan R. Liss, Inc., N.Y. (1987). The content of which is incorporated herein by reference.

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the following numerical value. In certain embodiments, the term about denotes a range of +/−10% of the thereafter following numerical value. In certain embodiments, the term about denotes a range of +/−5% of the thereafter following numerical value.

The terms “comprise(s),” “include(s),” “having,” “has,” “can,” “contain(s)” and variants thereof, as used herein, are intended to be open-ended transitional phrases, terms or words that do not preclude the possibility of additional acts or structures.

The term “comprising” also encompasses the term “consisting of”. The present disclosure also contemplates other embodiments “comprising”, “consisting of” and “consisting essentially of” the embodiments or elements presented herein, whether explicitly set forth or not.

The terms “empty recombinant AAV particle” and “empty rAAV”, which can be used interchangeably, denote a protein shell composed of adeno-associated capsid polypeptides without a therein encapsidated/packaged functional nucleic acid (rAAV=recombinant Adeno-Associated Virus particle). That is, an empty rAAV either may be free of encapsidated nucleic acid or comprises a nucleic acid or part thereof that is not transcribed at all or not transcribed into a functional transcript. Accordingly, an empty rAAV does not function to transfer a nucleic acid that encodes a functional protein or is transcribed into a functional transcript of interest into a target cell. In certain embodiments of all aspects and embodiments, the functional protein or the functional transcript of interest has a therapeutic effect.

The term “endogenous” denotes that something is naturally occurring, e.g. within a cell or naturally produced, e.g., by a cell.

The term “exogenous” denotes that something, e.g. a nucleotide sequence, does not originate from the same entity wherein it is present. For example, a nucleic acid is exogenous to a specific cell if it has been introduced into said cell by a DNA delivery method, such as, e.g., by transfection, electroporation, or transduction. Likewise, a nucleic acid is exogenous to an AAV particle if it is not originating from the same AAV particle or serotype. Thus, an exogenous nucleotide sequence is an artificial sequence, either in isolated form or within a cell or an rAAV, wherein the artificiality can originate, e.g., from the combination of subsequences of different origin, e.g. a combination of a recombinase recognition sequence with an SV40 promoter and a coding sequence of green fluorescent protein or a combination of AAV ITRs from a first serotype with the capsid polypeptides of a second serotype or a combination of AAV ITRs with a non-AAV nucleic acid, or from the deletion of parts of a sequence, e.g. a sequence coding only the extracellular domain of a membrane-bound receptor or a cDNA, or from the mutation of nucleobases in an endogenous nucleic acid sequence. This does not exclude that an “exogenous” nucleotide sequence may have an “endogenous” counterpart that is identical in base composition, but where the sequence is becoming an “exogenous” sequence by its combination with exogenous regulatory elements such as an exogenous secretion signal or promoter.

The terms “full recombinant AAV particle” or “full rAAV”, which can be used interchangeably, denote a non-covalent complex formed of a protein shell composed of adeno-associated capsid polypeptides and a therein encapsidated/packaged functional nucleic acid sequence. That is, a full rAAV comprises a nucleic acid that is transcribed into a functional transcript. Accordingly, the full rAAV functions to transfer a nucleic acid that encodes a protein or is transcribed into a transcript of interest into a target cell. In certain embodiments, a functional nucleic acid comprises at least one coding nucleic acid sequence interspaced between two adeno-associated viral inverted terminal repeats (ITRs).

The term “full to empty ratio” denotes the mathematical ratio of the number of full recombinant AAV particles (full rAAV) to the total number of recombinant AAV particles (sum of full rAAV and empty rAAV) in a sample or in a recombinant AAV particle preparation (rAAVp). As the number of full rAAV can be at most the same as the total number of rAAV, the ratio can be at most 1. Generally, the ratio is less than 1 and is expressed as a percentage. The number of full rAAV is determined by determining the number of nucleic acid sequences interspaced between two AAV ITRs in the sample or preparation. This can be done by PCR, especially digital droplet PCR (ddPCR) or quantitative PCR (qPCR). The total number of rAAV is determined by determining the number of protein shells formed of adeno-associated capsid polypeptides in the sample or preparation. This can be done by ELISA, especially by a capsid polypeptide specific ELISA.

The term “in-vitro” denotes either an artificial environment as such or that a process or reaction is performed within such an artificial environment.

The term “in-vivo” denotes the natural environment (e.g., an animal or a cell) of a compound or that a process or reaction is performed within its natural environment.

The terms “recombinant AAV vector” or “transgene”, which can be used interchangeably herein, denote a nucleic acid derived from a wild-type genome of an adeno-associated virus, wherein except for the ITR (adeno-associated virus Inverted Terminal Repeat) sequences all endogenous AAV nucleic acids are replaced by one or more exogenous nucleic acid(s). For example, such an exogenous nucleic acid can be a nucleic acid transcribed into a transcript of interest or that encodes a therapeutic protein or a therapeutic nucleic acid. Typically, for a recombinant AAV vector one or both ITR sequences of the wild-type AAV genome are retained. Thus, a recombinant AAV vector can be distinguished from a wild-type AAV vector, since all or at least a part of the viral genome has been replaced with a non-native (i.e. exogenous) nucleic acid with respect to the virus. Incorporation of a non-native nucleic acid therefore defines the AAV vector as a “recombinant” vector. It has to be pointed out that the serotype of the ITRs in the recombinant AAV vector does not need to be the same as the serotype of the adeno-associated capsid polypeptides forming the shell of the recombinant AAV particle comprising said recombinant AAV vector.

In principle, any non-AAV nucleic acid can be packaged into a shell composed of adeno-associated capsid polypeptides resulting in a “recombinant AAV particle”, e.g. for subsequent infection (transduction) of a cell, ex vivo, in vitro or in vivo.

As used herein, the term “serotype” is used to classify different wild-type and recombinant AAV particles based on the amino acid sequence of the polypeptides forming the protein shell (capsid) of the respective AAV particle. Originally, serologic distinctiveness was determined based on the lack of cross-reactivity between antibodies to one AAV particle as compared to another AAV particle. Such cross-reactivity differences are usually due to differences in capsid polypeptide sequences and the respective antigenic determinants (e.g., due to VP1, VP2, and/or VP3 sequence differences of AAV serotypes). Despite the possibility that AAV variants including capsid variants may not be serologically distinct from a reference or wild-type AAV or other AAV serotype, they differ by at least one amino acid residue compared to the reference or wild-type or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new AAV particle has no serological difference, this new AAV particle would be a subgroup or variant of the corresponding wild-type serotype. In many cases, serology testing for neutralizing activity has yet to be performed on mutant viruses with capsid sequence modifications to determine if they are of another serotype according to the traditional definition of serotype.

The term “vector” denotes the portion of a larger nucleic acid, e.g. of a recombinant plasmid, that is ultimately packaged or encapsulated or encapsidated either directly or in form of a single strand or in form of RNA into a protein shell composed of adeno-associated virus capsid polypeptides to form a recombinant AAV particle. In cases where recombinant plasmids are used to construct or manufacture recombinant AAV particles, the viral particle does not include the portion of the “plasmid” that does not correspond to the vector part of the recombinant plasmid. For example, in case of a rAAV the recombinant vector comprises that part of the recombinant plasmid that is interspaced between two AAV ITRs. The non-vector portion of the recombinant plasmid is referred to as the “plasmid backbone”. The plasmid backbone is important for cloning and amplification of the plasmid, a process that is needed for propagation and recombinant virus production, but is not itself packaged or encapsulated or encapsidated into the recombinant AAV particle. Thus, a “vector” refers to the nucleic acid that is packaged or encapsulated or encapsidated by a protein shell composed of adeno-associated virus capsid polypeptides, i.e. in a rAAV.

General Methods for Producing rAAV Particles

WO 1999/11764 reported methods for generating high titer helper-free preparations of recombinant AAV vectors. Not further defined AAV producer cells grown in suspension in bioreactors were infected with Adenovirus Type 5 (Ad5) at a multiplicity of infection (MOI) of 10 in low serum media at 1.5 L scale at different pH values. At a culture pH of 7.2 4.7 E+12 total particles were obtained, at a culture pH of 7.4 1.95 E+13 total particles were obtained, at a culture pH of 7.6 1.84 E+13 total particles were obtained and at a culture pH of 8.0 1.63 E+13 total particles were obtained. The cultivation was performed in a 1.5 L bioreactor and, thus, the cultivation volume can be calculated (75% of the nominal value) to have been about 1.125 L. Therefore the total particle number correspond to 4.2 E+09 vp/mL (pH 7.2), 1.7 E+10 vp/mL (pH 7.4), 1.6 E+10 vp/mL (pH 7.6) and 1.5 E+10 vp/mL (pH 8), respectively.

WO 2000/14205 reported the production of AAV particles in a non-defined cell type denoted as JL-14 cells by co-infection with adenoviral helper virus, whereby at a pH value of 7.4 the highest number of AAV particles (sum of intracellular and secreted AAV particles), at a pH value of 8 AAV particles with the highest infectivity and at a pH of 7.6 the highest ratio of number of AAV particles to infectivity was obtained. The cultivation was performed in a volume of 1.5 L medium and, thus, the total particle number correspond to 3.0 E+09 vp/mL (pH 7.2), 1.3 E+10 vp/mL (pH 7.4), 1.2 E+10 vp/mL (pH 7.6), 3.3 E+9 vp/mL (pH 7.8) and 1.1 E+10 vp/mL (pH 8), respectively. Based on the provided infectivity data it can be assumed that the full/empty ratio of the thereby produced rAAV particles is below 1%.

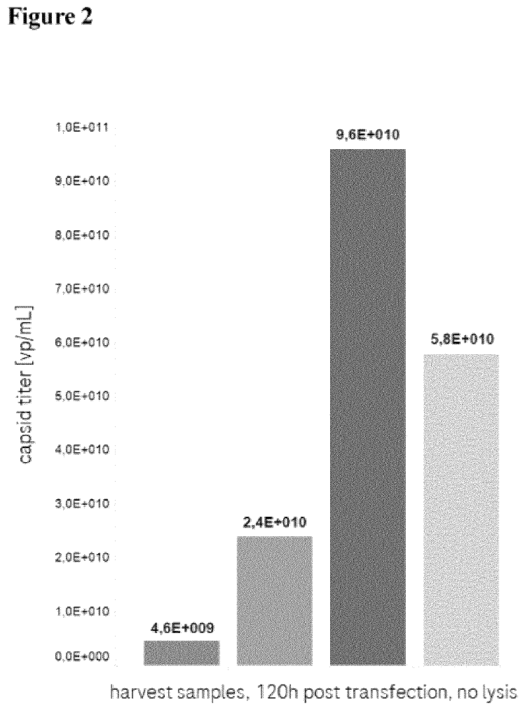

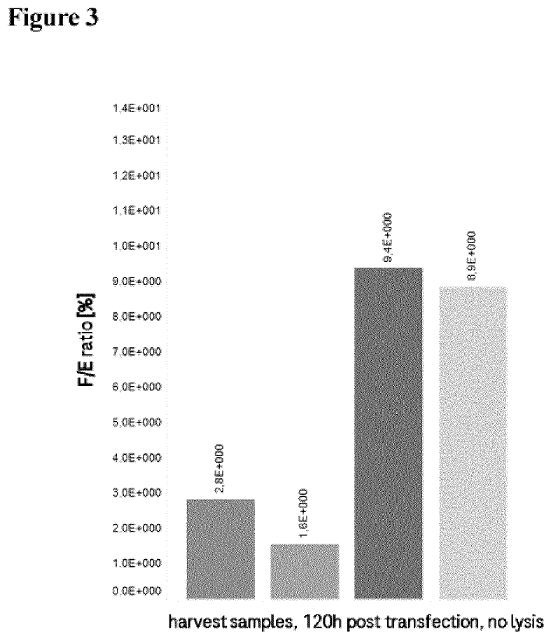

This is based on the following calculation of the data from FIGS. 2B and 3B of WO 2000/14205 (3 days post infection):

| day 3 pH | total DRPs (=vp) | =>vp/mL |
|---|---|---|
| 7.2 | 4.50E+12 | 3.00E+09 |
| 7.4 | 1.95E+13 | 1.30E+10 |
| 7.6 | 1.85E+13 | 1.23E+10 |
| 7.8 | 5.00E+12 | 3.33E+09 |
| 8 | 1.65E+13 | 1.10E+10 |

| day 3 pH | total RUs | =>RU/mL | =>RU/mL/vp/mL |
|---|---|---|---|
| 7.2 | 2.00E+09 | 1.33E+06 | 0.044% |
| 7.4 | 7.40E+09 | 4.93E+06 | 0.038% |
| 7.6 | 5.00E+09 | 3.33E+06 | 0.027% |
| 7.8 | 2.75E+09 | 1.83E+06 | 0.055% |
| 8 | 7.50E+09 | 5.00E+06 | 0.045% |

Piras, B. A., et al. (Mol. Ther. Meth. Clin. Dev. 3 (2016) 16015) compared distribution of AAV8 in cell culture media and lysates on days 3, 5, 6 and 7 post-transfection and found increasing viral production through day 6, with the proportion of viral particles in the media increasing from 76% at day 3 to 94% by day 7. Larger-scale productions showed that the ratio of full-to-empty AAV particles is similar in media and lysate, and that AAV harvested on day 6 post-transfection provides equivalent function in mice compared to AAV harvested on day 3. AAV-FVIII showed an increase in production when culture was extended from day 3 (1.1×1 E+13±9.2×1 E+11 and 3.6×1 E+13±2.5×1 E+12 total capsids in the lysate and media, respectively) to day 5 (6.7×1 E+12±6.7×1 E+11 and 4.5×1 E+13±2.6×1 E+12 total capsids in the lysate and media, respectively), day 6 (5.0×1 E+12±1.9×1 E+11 and 5.3×1 E+13±3.3×1 E+12 total capsids in the lysate and media, respectively), and day 7 (3.0×1 E+12±1.3×1 E+10 and 5.0×1 E+13±1.9×1 E+12 total capsids in the lysate and media, respectively). Piras et al. employed adherent HEK293T/17 cells cultured in Dulbecco's Modified Eagle's Medium with 10% fetal bovine serum supplemented with 2 mmol/l GlutaMAX (Life Technologies, Grand Island, NY). AAV was produced by two-plasmid transfection using PEIpro™ (Polyplus-transfection SA, Illkirch, France) 1 day after seeding cells at a density of 7.26×1 E+04 cells/cm².

Powers, A. D., et al. (Hum. Gene Ther. Meth. 27 (2016) 112-121) reported the development and optimization of AAV hFIX particle production by transient transfection in an iCELLis® fixed-bed bioreactor. A yield to as high as 9 E+14 viral particles per square meter of fixed bed were obtained. On day 3 after inoculation with HEK293T/17 cells, the vessel was transfected with plasmid scAAV-LP1-hFIXco-helpv3 and plasmid CR21+LTAAV help 2-8 at a plasmid mass ratio of 3:1, respectively, using polyethylenimine (PEIpro™ Transfection Reagent Cat #115-375; Polyplus) in IMIDM (Lonza) or DMEM supplemented with 10% FBS and 6 mM GlutaMAX™. The PEI and DNA solutions were combined at a 2:1 ratio.

Poulain, A., et al. (j. Biotechnol. 255 (2017) 16-27 reported rapid protein production from stable CHO cell pools using plasmid vector and the cumate gene-switch. Cells were transfected using linear polyethylenimine (PEIpro™) from Polyplus-Transfection (Illkirch, France). On the day of transfection, cells were suspended at a density of 2×1 E+06 cells/mL in CD DG44 medium (Life Technologies Inc., Burlington, ON, Canada), supplemented with 4 mM glutamine and 0.1% Kolliphor® P 188. The cell suspension was distributed in 6-well plates (1.8 mL/well). The DNA:PEIpro™ complexes were prepared at a ratio of 1:5 (w:w), with a total of 2 μg DNA per well to transfect in 100 L of complete culture medium.

WO 2017/096039 reported scalable methods for producing recombinant AAV vectors in serum-free suspension cell culture systems suitable for clinical use. Production of rAAV vectors was performed in bioreactors with HEK293F cells using triple transfection at a cell density of 1 E+06 cells/mL (1.000.000 cells/mL) with a plasmid ratio of 1:1:1 and a PEI-based transfection reagent (PEI/DNA weight ratio of 2:1 with 12 of PEI as free PEI) at a temperature of 37° C. and a pH value of 7.2.

Nyamay'antu, A., et al. (Cell Gen. Ther. Ins. 4 (2018) 71-79) reported that PEI is widely used due to its affordability and high DNA delivery efficiency, in both adherent and suspension cells grown in serum-free medium. PEIpro™ is suited for small- to large-scale production of various viruses, notably AAV particles. In stirred-tank bioreactors using HEK293 or HEK293T cells titers in the range of 0.8-1.5 E+09−E+10 vg/mL can be obtained.

Koo, T., et al. (Nat. Commun. 9 (2018) 1855) reported that CRISPR-LbCpf1 prevents choroidal neovascularization in a mouse model of age-related macular degeneration. To produce AAV vectors, they were pseudotyped in AAV9 capsids. HEK293T cells (ATCC, CRL-3216) were transfected with pAAV-ITR-LbCpf1-crRNA, pAAV2/9 encoding for AAV2rep and AAV9cap, and helper plasmid. HEK293T cells were cultured in DMEM with 2% FBS. Recombinant pseudotyped AAV vector stocks were generated using PEI coprecipitation with PEIpro™ (Polyplus-transfection) and triple-transfection with plasmids at a molar ratio of 1:1:1 in HEK293T cells. After 72 h of incubation, cells were lysed and particles were purified by iodixanol step-gradient ultracentrifugation.

The Rep proteins from AAV2 are commonly and nearly exclusively used in the production of rAAVs derived from the serotypes AAV1 to AAV13 (Daya, S., and Berns, K. I., Clin. Microbiol. Rev. 21 (2008) 583-593; Zincarelli, C., et al., Mol. Ther. 16 (2008) 1073-1080).

WO 2019/094253 reported means and methods for preparing viral vectors and uses thereof. Adherent HEK293 cells were cultivated in bioreactors at a pH value of 7.23 and triple transfected (plasmid ratio 1:1:1) with PEI/DNA at a PEI-plasmid ratio of about 1:1 by weight.

Collaud, F. et al. (Mol. Ther. Meth. Clin. Dev. 12 (2019) 157-174) reported titers for rAAV8 particles of 6.0±1.89 E+04 vg/cell and 1.77±1.37 E+04 vg/cell for (single stranded) and (self-complementary) AAV, respectively, for adherent HEK293 cells. A fully scalable method based on triple transfection of HEK293 cells cultured in suspension was also reported. Triple transfection of HEK293 cells was performed with polyethylenimine (PEIpro™, Polyplus) directly in 10 L bioreactors. AAV vectors were recovered from both supernatant and cells by mild detergent lysis followed by AVB Sepharose affinity column purification. Purified vectors were then concentrated and tested for quality and potency. No information about the pH value and obtained titers are provided.

Nyamay'antu, A., et al. (Cell Gen. Ther. Ins. 6 (2020) 655-661) reported that the efficiency of the delivery process is essential to obtain a high number of producing cells. Of the existing transfection methods, the use of PEI-based transfection reagent is predominant in gene therapy as it combines affordability and compatibility for transfection of adherent and suspension cells. In comparison to the gold standard PEIpro™ used for viral vector manufacturing, FectoVIR™-AAV has been found to improve significantly rAAV2 production yield of both viral genome production and packaging efficiency in suspension cells of an rAAV2-GFP of up to 10-fold compared to PEIMax™ and up to 2-fold compared to PEIpro™, respectively, when each transfection reagent is used under the recommended conditions. In more detail, suspension HEK293T cells were transfected using the respective transfection reagent under the recommended conditions. rAAV2-GFP were harvested 72 hours post transfection. The obtained titer with VectoVIR™ is in the range of 1 E+04 to 4.5 E+04 vg/cell depending on the used volume of complexation (1%-10%) corresponding to 1 E+12 vg/mL. The respective functional titers are about 2-8 E+08 TU/mL. The results are almost independent of the employed cultivation medium.

In a blog article entitled “Optimization of AAV production for high-yielding and scalable GMP processes with Catalent” (www.polyplus-transfection.com) different transfection reagent to DNA ratios were tested with the two serotypes AAV9 (1:1 and 2:1) and AAV2 (3:1.5 and 5:2.5). The AAV2 vector yield was not affected as notably, with a 4-5-fold increase in the vector genome titer and a 3-6-fold increase in the viral particle titer with FectoVIR™-AAV as compared to PEIpro™. These results show that improvement in yield may vary with the AAV serotype. In a further study comparing additional AAV2 and AAV5 vectors (different from the previous AAV2 and AAV5 vectors) and using a DoE approach to optimization, experiments were conducted varying transfection reagent to DNA ratios (3:2, 3:1.5) and plasmid DNA molar ratios (1:1:1, 2:1:2, 1:2:1) were performed. A 3-5-fold increase for AAV2 and a 1.1-1.6-fold increase for AAV5 in the vector genome titer with FectoVIR™-AAV compared to PEIpro™ was observed. The viral particle titer increased 3.5-4.5-fold for AAV2 and 2.5-3.75-fold for AAV5. Reagent-to-DNA ratios of 2:1 and 1.5:1 and plasmid ratios of 1:1:1 to 2:1:2 to 1:2:1 were used. The obtained titer with VectoVIR™ was in the range of 4 E+11 to 1 E+12 vg/mL.

Rossi, A. and Peigné, C-M. (Cell Culture Dish Article May 17, 2021) outlined that, typically, AAV production titers are around 1 E+11 to 1 E+12 in vg/mL and 1 E+08 to 1 E+09 TU/mL.

To put the numbers into perspective, it is important to keep in mind that two AAV serotypes will most likely not give the same yield, even when using the same production process.

In more detail, AAV production yields vary depending on the serotype and the gene of interest. Generally, to increase production of a given AAV the parameters that directly have an impact on the yield are optimized: plasmid DNA, transfection reagent, cells and medium. PEI-based transfection processes, e.g., can decrease down by 10 times DNA amount and can be used to transfect cells grown in the presence of or in the absence of serum.

Wosnitzka, K., et al. (Cell Gen. Ther. Ins. 7 (2021) 1-7) reported that analysis of physical titers revealed a 3-fold increase in both viral particles (VP) and viral genome (VG) per ml of cell culture when using FectoVIR™-AAV transfection reagent compared to PEIpro™.

Porte, M., et al. (poster entitled “Next-Generation Transfection Reagent for Large Scale AAV Manufacturing”, Polyplus, Illkirch, France) reported the transfection of suspension-HEK293T cells with the optimal conditions for the other PEI-based reagent (1.5 g/million cells, ratio DNA:PEI of 1 μg:4 μL) and FectoVIR™-AAV (1 g/million cells, ratio DNA:reagent of 1 μg: 1 μL) following the recommended protocol for each reagent. A titer of about 5 E+11 vg/mL versus 1.5 E+11 vg/mL using FectoVIR™ and PEI-based transfection reagent, respectively, with a packaging efficiency of 20% vs. about 13.5%, respectively, was obtained.

### Recombinant Cell

Generally, for efficient as well as large-scale production of a rAAV a cell expressing and, if possible, also secreting said rAAV is used. Such a cell is termed “recombinant producer cell” or short “producer cell”.

For the generation of a recombinant producer cell a suitable mammalian cell is transfected with the nucleic acids required for producing said rAAV, including the required AAV helper functions.

Generally, for expression of a coding sequence, i.e. of an open reading frame, additional regulatory elements, such as a promoter and a polyadenylation signal (sequence), are necessary. Thus, for functional transcription an open reading frame has to be and is operably linked to said additional regulatory elements. This can be achieved by combining these parts into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case of an rAAV, which is composed of different (monomeric) capsid polypeptides and a therein encapsidated single stranded DNA molecule and which in addition requires other viral helper functions for production and encapsidation, a multitude of expression cassettes differing in the contained open reading frames/coding sequences are required. In this case, at least an expression cassette for each of the transgene, for the polypeptides forming the capsid of the rAAV, for the required viral helper functions are required. Thus, individual expression cassettes at least for each of the helper functions E1A, E1B, E2A, E4orf6, the rep and cap genes are required. HEK293 cells express the E1A and E1B helper functions constitutively.

### Adeno-Associated Virus (AAV)

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV vectors are described (see also WO 93/24641). Additional references describing AAV vectors are West et al., Virol. 160 (1987) 38-47; Kotin, Hum. Gene Ther. 5 (1994) 793-801; and Muzyczka J. Clin. Invest. 94 (1994) 1351. Construction of recombinant AAV vectors is described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828.

An AAV is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin provided that the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV2 other integration sites have been found, such as, e.g., on chromosome 5 [(Chr) 5 (p13.3)], termed AAVS2, and on chromosome 3 [(Chr) 3 (p24.3)], termed AAVS3.

AAVs are categorized into different serotypes. These have been allocated based on parameters, such as hemagglutination, tumorigenicity and DNA sequence homology. Up to now, more than 12 different serotypes and more than a hundred sequences corresponding to different clades of AAV have been identified.

The capsid protein type and symmetry determines the tissue tropism of the respective AAV. For example, AAV2, AAV4 and AAV5 are specific to retina, AAV2, AAV5, AAV8, AAV9 and AAV-rh.10 are specific for brain, AAV1, AAV2, AAV6, AAV8 and AAV9 are specific for cardiac tissue, AAV1, AAV2, AAV5, AAV6, AAV7, AAV8, AAV9 and AAV10 are specific for liver, AAV1, AAV2, AAV5 and AAV9 are specific for lung.

Pseudotyping denotes a process comprising the cross packaging of the AAV genome between various serotypes, i.e. the genome is packaged with differently originating capsid proteins.

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding open reading frame provides for four proteins of different size, which are termed Rep78, Rep68, Rep52 and Rep40. These are involved in replication, rescue and integration of the AAV. The Cap protein encoding open reading frame provides four proteins, which are termed VP1, VP2, VP3, and AAP. VP1, VP2 and VP3 are part of the proteinaceous capsid of the AAV particles. The combined rep and cap open reading frames are flanked at their 5′- and 3′-ends by so-called inverted terminal repeats (ITRs). For replication, an AAV requires in addition to the Rep and Cap proteins the products of the genes E1A, E1B, E4orf6, E2A and VA of an adenovirus or corresponding factors of another helper virus.

In the case of an AAV of the serotype 2 (AAV2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The wild-type AAV genome harbors three transcription promoters P5, P19, and P40 (Laughlin et al., Proc. Natl. Acad. Sci. USA 76 (1979) 5567-5571) for the expression of the rep and cap genes.

The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome an in trans organization of the rep and cap gene products is required.

The rep gene locus comprises two internal promoters, termed P5 and P19. It comprises open reading frames for four proteins. Promoter P5 is operably linked to a nucleic acid sequence providing for non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The two larger Rep proteins, Rep78 and Rep68, are essential for AAV duplex DNA replication, whereas the smaller Rep proteins, Rep52 and Rep40, seem to be essential for progeny and single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene locus comprises one promoter, termed P40. Promoter P40 is operably linked to a nucleic acid sequence providing for 2.6 kb mRNA, which, by alternative splicing and use of alternative start codons, encodes the Cap proteins VP1 (87 kDa, non-spliced mRNA transcript), VP2 (72 kDa, from the spliced mRNA transcript), and VP3 (61 kDa, from alternative start codon). VP1 to VP3 constitute the building blocks of the viral capsid. The capsid has the function to bind to a cell surface receptor and allow for intracellular trafficking of the virus. VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is contained. This may be either the “plus” or “minus” strand. AAV particles containing a DNA molecule are infectious. Inside the infected cell, the parental infecting single stranded DNA is converted into a double stranded DNA, which is subsequently amplified. The amplification results in a large pool of double stranded DNA molecules from which single strands are displaced and packaged into capsids.

Adeno-associated viral (AAV) vectors can transduce dividing cells as well as resting cells. It can be assumed that a transgene introduced using an AAV vector into a target cell will be expressed for a long period. One drawback of using an AAV vector is the limitation of the size of the transgene that can be introduced into cells.

Parvovirus particles, including AAV serotypes and variants thereof, provide a means for ex vivo, in vitro and in vivo delivery of nucleic acid, which encode proteins, into cells such that the infected cells express the encoded protein. AAVs are viruses useful as gene therapy vectors as they can penetrate cells and introduce nucleic acid/genetic material so that the nucleic acid/genetic material may be stably maintained in the infected cells. Because AAV are not associated with pathogenic disease in humans, AAVs are able to deliver heterologous polynucleotide sequences (e.g., therapeutic proteins and agents) to human patients without causing substantial AAV-related pathogenesis or disease.

AAV particles used as vehicles for effective gene delivery possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

Recombinant AAV particles do not typically include viral genes associated with pathogenesis. Such particles typically comprise a genome, wherein one or more of the wild-type AAV genes have been deleted in whole or in part, for example, rep and/or cap genes, but retain at least one functional flanking ITR sequence, as necessary for the rescue, replication, and packaging of the recombinant vector into an rAAV. Thus, an AAV vector includes sequences required in cis for replication and packaging (i.e. functional ITR sequences).

Recombinant AAV particles, as well as methods and uses thereof, can be based on any wild-type AAV genome or serotype or combination thereof. As a non-limiting example, a rAAV can be based upon any wild-type AAV genome, i.e. comprise the respective ITR sequences, such as AAV1, -2, -3, -4, -5, -6, -7, -8, -9, -10, -11, -12, 2i8, rh.74, rh.10 or 7m8 for example. Such particles can be based on the same strain or serotype (or subgroup or variant), or be different from each other. As a non-limiting example, a rAAV based upon one wild-type genome can be identical or different to one or more of the capsid proteins that package the vector. In addition, a recombinant AAV vector can be based upon an AAV (e.g., AAV2) wild-type serotype genome distinct from one or more of the AAV capsid proteins that package the vector. For example, the AAV vector can be based upon AAV2, whereas at least one of the three capsid proteins could be an AAV1, AAV3, AAV4, AAV5, AAV6, AAV7, AAV8, AAV9, AAV10, AAV 11, AAV12, AAV-2i8, AAV-rh.74, AAV-rh.10 or AAV-7m8 or a variant thereof, for example. AAV variants include variants and chimeras of AAV1, AAV2, AAV3, AAV4, AAV5, AAV6, AAV7, AAV8, AAV9, AAV10, AAV 11, AAV12, AAV-2i8, AAV-rh.74, AAV-rh.10 and AAV-7m8 capsids.

In certain embodiments of all aspects and embodiments of the invention, the rAAV particle is derived from a wild-type AAV particle selected from the group consisting of AAV1, AAV2, AAV3, AAV4, AAV5, AAV6, AAV7, AAV8, AAV9, AAV10, AAV11, AAV12, AAV-2i8, AAV-rh.74, AAV-rh.10 and AAV-7m8, as well as variants (e.g., capsid variants, such as amino acid insertions, additions, substitutions and deletions) thereof, for example, as set forth in WO 2013/158879, WO 2015/013313 and US 2013/0059732 (disclosing LK01, LK02, LK03, etc.).

In certain embodiments of all aspects and embodiments of the invention, the rAAV comprises a capsid polypeptides with an amino acid sequence having 70% or more sequence identity to an wild-type AAV1, AAV2, AAV3, AAV4, AAV5, AAV6, AAV7, AAV8, AAV9, AAV10, AAV11, AAV12, AAV-2i8, AAV-rh.10, AAV-rh.74, or AAV-7m8 capsid sequence.

In certain embodiments of all aspects and embodiments of the invention, the rAAV particle comprises one or two ITR sequence having 70% or more sequence identity to a wild-type AAV1, AAV2, AAV3, AAV4, AAV5, AAV6, AAV7, AAV8, AAV9, AAV10, AAV11 or AAV12 ITR sequence.

Recombinant AAV particles can be incorporated into pharmaceutical compositions. Such pharmaceutical compositions are useful for, among other things, administration and delivery to a subject in vivo or ex vivo. In certain embodiments, the pharmaceutical composition contains a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744, which are incorporated herein by reference in their entirety.

Recombinant Adeno-Associated Viral Particles (rAAV Particles)

Different methods are known in the art for generating recombinant AAV particles. For example, transfection with an AAV vector comprising plasmid and a plasmid comprising AAV helper sequences (rep and cap) in conjunction with co-infection with one AAV helper virus (e.g., adenovirus, herpesvirus, or vaccinia virus) or transfection with a recombinant AAV vector comprising plasmid, an AAV helper plasmid (comprising rep and cap), and an helper function plasmid. Non-limiting methods for generating rAAV are described, for example, in U.S. Pat. Nos. 6,001,650, 6,004,797, WO 2017/096039, and WO 2018/226887. Following rAAV production (i.e. particle generation in cell culture systems), rAAV can be obtained from the host cells and/or cell culture supernatant and purified.

For the generation of recombinant AAV particles, expression of the Rep and Cap proteins, the helper proteins E1A, E1B, E2A and E4orf6 as well as optionally the adenoviral VA RNA in a single mammalian cell is required. The helper proteins E1A, E1B, E2A and E4orf6 can be expressed using any promoter as shown by Matsushita et al. (Gene Ther. 5 (1998) 938-945), especially the CMV IE promoter. Thus, any promoter can be operably linked to said genes for functional expression.

Generally, to produce rAAV, different, complementing plasmids are co-transfected into a host cell. One of the plasmids comprises the transgene sandwiched between the two cis acting AAV ITRs. The missing AAV elements required for replication and subsequent packaging of progeny recombinant genomes, i.e. the open reading frames for the Rep and Cap proteins, are contained in trans on a second plasmid. The overexpression of the Rep proteins results in inhibitory effects on cell growth (Li, J., et al., J. Virol. 71 (1997) 5236-5243). Additionally, a third plasmid comprising the genes of a helper virus, i.e. E1, E4orf6, E2A and VA from adenovirus, is required for rAAV production.

To reduce the number of required plasmids, rep, cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrid vectors can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

In order to limit the transgene activity to specific tissues, i.e. to limit the site of action, the transgene can be operably linked to an inducible or tissue specific promoter (see, e.g., Yang, Y., et al. Hum. Gene. Ther. 6 (1995) 1203-1213).

The coding sequences of E1A and E1B (open reading frames) can be derived from a human adenovirus, such as, e.g., in particular of human adenovirus serotype 2 or serotype 5. An exemplary sequence of human Ad5 (adenovirus serotype 5) is found in GenBank entries X02996, AC_000008 and that of an exemplary human Ad2 in GenBank entry AC_000007. Nucleotides 505 to 3522 comprise the nucleic acid sequences encoding E1A and E1B of human adenovirus serotype 5. Plasmid pSTK146 as reported in EP 1 230 354, as well as plasmids pGS119 and pGS122 as reported in WO 2007/056994, can also be used as a source for the E1A and E1B open reading frames.

E1A is the first viral helper gene that is expressed after adenoviral DNA enters the cell nucleus. The E1A gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will die (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis. The E1B 19 kDa protein is involved in the prevention of E1A-induced apoptosis of cells.

The E2 gene encodes different proteins. The E2A transcript codes for the single strand-binding protein (SSBP), which is essential for AAV replication

In addition, the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

The viral associated RNA (VA RNA) is a non-coding RNA of adenovirus (Ad), regulating translation. The adenoviral genome comprises two independent copies: VAI (VA RNAI) and VAII (VA RNAII). Both are transcribed by RNA polymerase III (see, e.g., Machitani, M., et al., J. Contr. Rel. 154 (2011) 285-289) from a type 2 polymerases III promoter. For recombinant AAV particle production, the adenoviral VA RNA gene can be driven by any promoter.

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes. Said disclosure is herewith incorporated by reference in its entirety into the current application.

VA RNAs, VAI and VAII, are consisting of 157-160 nucleotides (nt).

Depending on the serotype, adenoviruses contain one or two VA RNA genes. VA RNAI is believed to play the dominant pro-viral role, while VA RNAII can partially compensate for the absence of VA RNAI (Vachon, V. K. and Conn, G. L., Virus Res. 212 (2016) 39-52).

The VA RNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

Maekawa, A., et al. (Nature Sci. Rep. 3 (2013) 1136) reported efficient production of adenovirus vector lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI corresponds to nucleotides 10586-10810 of GenBank entry AC_000007 sequence. The human adenovirus 5 VA RNAI corresponds to nucleotides 10579-10820 of GenBank entry AC_000008 sequence.

### General Description of Recombinant AAV Particle Production

After entry into the host cell nucleus, AAV can follow either one of two distinct and interchangeable pathways of its life cycle: the lytic or the lysogenic. The former develops in cells infected with a helper virus such as Ad or herpes simplex virus (HSV) whereas the latter is established in host cells in the absence of a helper virus. When a latently infected cell is super-infected with a helper virus, the AAV gene expression program is activated leading to the AAV Rep-mediated rescue (i.e., excision) of the provirus DNA from the host cell chromosome followed by replication and packaging of the viral genome. Finally, upon helper virus-induced cell lysis, the newly assembled virions (particles) are released. Thus, the lytic phase of the AAV life cycle is induced.

Therefore, in the presence of Ad helper functions, the rAAV vector is subjected to the wild-type AAV lytic processes by being rescued from the plasmid backbone, replicated and packaged into preformed AAV capsids as single-stranded molecules (Gonçalves, M. A. F. V., Virol. J., 2 (2005) 43).

Generation of a recombinant AAV particle involves replacing a majority of the AAV's wild-type genome with a desired transgene and providing the viral genes that are essential for virus packaging in-trans on a separate plasmid. Once all components are transfected together into a packaging cell line, recombinant AAV particles are assembled using the cell's cellular machineries. The process of viral assembly and encapsulation takes roughly two days, after which the cells are lysed to release the rAAV for further purification and concentration (https.//old.abmgood.com/marketing/knowledge_base/Adeno_Associated_Virus_Production_and_Modification_of_AAV.php).

AAV is not released very efficiently from the cells, although major differences have been observed between serotypes (see, e.g., Strobel, B., et al., Lamla T. Comparative Analysis of Cesium Chloride- and Iodixanol-Based Purification of Recombinant Adeno-Associated Viral Vectors for Preclinical Applications. Hum. Gene Ther. Methods 26 (2015) 147-157). When harvesting the culture, a cell disruption method is usually applied to recover the vectors entrapped in the cells.

Historically, manufacturing of rAAVs was performed by double transfection of a plasmid containing the rep and the cap ORFs and a plasmid with the gene of interest flanked by ITRs. Then, a helper virus, typically Adenovirus, was co-infected (see, e.g., Aponte-Ubillus, J. J., et al., Appl. Microbiol. Biotechnol. 102 (2018) 1045-1054; Muzyczka, N., Curr. Top. Microbiol. Immunol. 158 (1992) 97-129). In this setting, the separation of the helper virus from the final product was difficult, but a critical element to avoid induction of inflammatory responses after injection into patients (see, e.g., Schnell, M. A., et al., Mol. Ther. 3 (2001) 708-722.). Therefore, production of rAAVs nowadays moved towards an adenovirus-free approach by utilizing triple transfection (see, e.g., Large, E. E., et al., Viruses 13 (2021) 1336). To this end, three components are needed: one plasmid encoding the genes for Rep and Cap without the ITRs, a second plasmid with the transgene of interest flanked by ITRs, and a helper plasmid to provide the helper genes of the helper virus (see, e.g., Aponte-Ubillus, J. J., et al., Appl. Microbiol. Biotechnol. 102 (2018) 1045-1054; Farris, K. D. and Pintel, D. J., Hum. Gene Ther. 19 (2008) 1421-1427; Grimm, D., et al., Hum. Gene Ther. 9 (1998) 2745-2760; Ferrari, F. K., et al., Nat. Med. 3 (1997) 1295-1297). For example, the Adenovirus helper bears the minimal required adenoviral genes E2A, E4 and VA. It is important to note, that the Human Embryonic Kidney cells 293 (HEK293) constitutively express the adenoviral genes E1A/B, which are also required for production of rAAVs. Therefore, HEK293 cells are classic producer cells for rAAVs and for manufacturing. Other cell types require a supplementation of E1A/B.

Carter et al. have shown that the entire rep and cap open reading frames in the wild-type AAV genome can be deleted and replaced with a transgene (Carter, B. J., in “Handbook of Parvoviruses”, ed. by P. Tijssen, CRC Press, pp. 155-168 (1990)). Further, it has been reported that the ITRs have to be maintained to retain the function of replication, rescue, packaging, and integration of the transgene into the genome of the target cell.

When cells comprising the respective viral helper genes are transduced by an AAV vector, or, vice versa, when cells comprising an integrated AAV provirus are transduced by a suitable helper virus, then the AAV provirus is activated and enters a lytic infection cycle again (Clark, K. R., et al., Hum. Gene Ther. 6 (1995) 1329-1341; Samulski, R. J., Curr. Opin. Genet. Dev. 3 (1993) 74-80).

Producer cells contain the rep and cap gene sequences, as well as the transgene cassette flanked by ITR sequences on one or more plasmids that are retained, e.g., via drug selection. Production of rAAV in these cell lines generally occurs after their infection with the required helper functions. Therefore, cells are infected either with replication-competent adenovirus (usually wild type Ad5) or a plasmid comprising the respective helper genes to supply helper virus proteins and initiate rAAV production. A packaging cell line differs from a producer cell line as it only contains the rep and cap genes.

More generally, cells transfected or transduced with DNA for the recombinant production of AAV particles can be referred to as a “recombinant cell”. Such a cell can be any mammalian cell that has been used as recipient of a nucleic acid (plasmid) encoding packaging proteins, such as AAV packaging proteins, a nucleic acid (plasmid) encoding helper proteins, and a nucleic acid (plasmid) that encodes a protein or is transcribed into a transcript of interest, i.e. a transgene placed between two AAV ITRs. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth media appropriate for sustaining cell viability or providing cell growth and/or proliferation are commercially available. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such media can be supplemented with vitamins and/or trace minerals and/or salts and/or amino acids, such as essential amino acids for mammalian (e.g., human) cells.

For producing rAAV, three plasmids are co-transfected into a mammalian cell. The transgene plasmid encodes the expression cassette, which is cloned between the AAV ITRs, whereas rep and cap genes are provided in trans by co-transfecting a second, packaging plasmid (rep/cap plasmid) to ensure AAV replication and packaging. The third plasmid, also referred to as helper plasmid, contains the minimal helper virus factors, commonly adenoviral E2A, E4orf6 and VA genes, but lacking AAV ITRs.

Diverse methods for the DNA transfer into mammalian cells have been reported in the art. These are all useful in the methods according to the current invention. In certain embodiments of all aspects and embodiments, electroporation, nucleofection, or microinjection for nucleic acid transfer/transfection is used. In certain embodiments of all aspects and embodiments, an inorganic substance (such as, e.g., calcium phosphate/DNA co-precipitation), a cationic polymer (such as, e.g., polyethylenimine, DEAE-dextran), or a cationic lipid (lipofection) is used for nucleic acid transfer/transfection is used. Calcium phosphate and polyethylenimine are the most commonly used reagents for transfection for nucleic acid transfer in larger scales (see, e.g., Baldi et al., Biotechnol. Lett. 29 (2007) 677-684), whereof polyethylenimine is preferred.

The growth in serum-free suspension culture and improvement of efficiency and reproducibility of transfection conditions using PEI as a transfection reagent permits ready scale-up the AAV production using shake-flasks, wave, or stirred-tank bioreactors.

The composition may comprise further plasmids or/and cells. Such plasmids and cells may be in contact with free PEI.

In addition to PEI, valproic acid (VPA) can be used to improve transfection efficiency. VPA, a branched short-chain fatty acid and inhibits histone deacetylase activity. Due to this reason, it is commonly added to mammalian cell culture as an enhancer of recombinant protein production.

Encoded AAV packaging proteins include, in certain embodiments of all aspects and embodiments, AAV rep and/or AAV cap. Such AAV packaging proteins include, in certain embodiments of all aspects and embodiments, AAV rep and/or AAV cap proteins of any AAV serotype.

Encoded helper proteins include, in certain embodiments of all aspects and embodiments, adenovirus E1A and E1B, adenovirus E2 and/or E4, VA RNA, and/or non-AAV helper proteins.

The cultivation can be performed using the generally used conditions for the cultivation of eukaryotic cells of about 37° C., 95% humidity and 8 vol.-% CO₂. The cultivation can be performed in serum containing or serum free medium, in adherent culture or in suspension culture. The suspension cultivation can be performed in any fermentation vessel, such as, e.g., in stirred tank reactors, wave reactors, rocking bioreactors, shaker vessels or spinner vessels or so called roller bottles. Transfection can be performed in high throughput format and screening, respectively, e.g. in a 96 or 384 well format.

Methods according to the current invention can include AAV particles of any serotype, or a variant thereof. In certain embodiments of all aspects and embodiments, a recombinant AAV particle comprises any of AAV serotypes 1-12, an AAV VP1, VP2 and/or VP3 capsid protein, or a modified or variant AAV VP1, VP2 and/or VP3 capsid protein, or wild-type AAV VP1, VP2 and/or VP3 capsid protein. In certain embodiments of all aspects and embodiments, an AAV particle comprises an AAV serotype or an AAV pseudotype, where the AAV pseudotype comprises an AAV capsid serotype different from an ITR serotype.

Expression control elements include constitutive or regulatable control elements, such as a tissue-specific expression control element or promoter.

ITRs can be any of AAV2 or AAV6 or AAV8 or AAV9 serotypes, or a combination thereof. AAV particles can include any VP1, VP2 and/or VP3 capsid protein having 75% or more sequence identity to any of AAV1, AAV2, AAV3, AAV4, AAV5, AAV6, AAV10, AAV11, AAV12, AAV 2i8, AAV rh.10, AAV rh.74 or AAV 7m8 VP1, VP2 and/or VP3 capsid proteins, or comprises a modified or variant VP1, VP2 and/or VP3 capsid protein selected from any of AAV1, AAV2, AAV3, AAV4, AAV5, AAV6, AAV8, AAV9, AAV-2i8, AAV-rh.10, AAV-rh.74 and AAV-7m8 AAV serotypes.

Following production of recombinant viral (e.g., AAV) particles, if desired, the viral (e.g., rAAV) particles can be purified and/or isolated from host cells using a variety of conventional methods. Such methods include column chromatography, CsCl gradients, iodixanol gradient and the like.

For example, a plurality of column purification steps such as purification over an anion exchange column, an affinity column and/or a cation exchange column can be used. (See, e.g., WO 02/12455 and US 2003/0207439). Alternatively, or in addition, an iodixanol or CsCl gradient steps can be used (see, e.g., US 2012/0135515; and US 2013/0072548). Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

An objective in the rAAV production and purification systems is to implement strategies to minimize/control the generation of production related impurities such as proteins, nucleic acids, and vector-related impurities, including wild-type/pseudo wild-type AAV species (wtAAV) and AAV-encapsulated residual DNA impurities.

Considering that the rAAV represents only a minor fraction of the biomass, rAAV need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

In certain embodiments of all aspects and embodiments of the method according to the current invention, as an initial step, typically the cultivated cells that produce the rAAV particles are harvested, optionally in combination with harvesting cell culture supernatant (medium) in which the cells (suspension or adherent) producing recombinant AAV particles have been cultured. The harvested cells and optionally cell culture supernatant may be used as is, as appropriate, lysed or concentrated. Further, if infection is employed to express helper functions, residual helper virus can be inactivated. For example, adenovirus can be inactivated by heating to temperatures of approximately 60° C. for, e.g., 20 minutes or more, which inactivates only the helper virus since AAV is heat stable while the helper adenovirus is heat labile.

The cells in the harvested cultivation broth can be lysed using methods now in the art, such as, e.g., detergent lysis or freeze-thaw cycles, to release the rAAV particles. Concurrently during cell lysis or subsequently after cell lysis, a nuclease, such as, e.g., benzonase, is added to degrade contaminating DNA. Typically, the resulting lysate is clarified to remove cell debris, e.g. by filtering or centrifuging, to render a clarified cell lysate. In a particular example, the lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains recombinant AAV particles (comprising full as well as empty rAAVs) and production/process related impurities, such as soluble cellular components from the host cells that can include, inter alia, cellular proteins, lipids, and/or nucleic acids, and cell culture medium components. The optionally clarified lysate is then subjected to purification steps to purify the rAAV (comprising rAAV vectors) from impurities using chromatography. The clarified lysate may be diluted or concentrated with an appropriate buffer prior to the first chromatography step.

After cell lysis, optional clarifying, and optional dilution or concentration, a plurality of subsequent and sequential chromatography steps can be used to purify the rAAV.

The first chromatography step is preferably an affinity chromatography step using an AAV affinity chromatography ligand.

If the first chromatography step is affinity chromatography the second chromatography step can be anion exchange chromatography. Thus, in certain embodiments of all aspects and embodiments, rAAV purification is via affinity chromatography, followed by purification via anion exchange chromatography or/and cation exchange chromatography or/and size exclusion chromatography, in any order or sequence or combination.

The removal of empty capsids from full ones, for example, during downstream processing is based on their different isoelectric points (pI) in anion exchange chromatography. The average calculated pI across all serotypes is 5.9 for full capsids and 6.3 for empty capsids (Venkatakrishnan, B., et al., J. Virol. 87 (2013) 4974-4984).

Cation exchange chromatography functions to separate the AAV from cellular and other components present in the clarified lysate and/or column eluate from an affinity or size exclusion chromatography. Examples of strong cation exchange resins capable of binding rAAV over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, MA, USA). Additional examples include Capto S, Capto S ImpAct, Capto S ImpRes (strong cation exchangers available from GE Healthcare, Marlborough, MA, USA), and commercial DOWEX®, AMBERLITE®, and AMBERLYST® families of resins available from Aldrich Chemical Company (Milwaukee, WI, USA). Weak cation exchange resins include, without limitation, any carboxylic acid based resin. Exemplary cation exchange resins include carboxymethyl (CM), phospho (based on the phosphate functional group), methyl sulfonate (S) and sulfopropyl (SP) resins.

Anion exchange chromatography functions to separate rAAV from proteins, cellular and other components present in the clarified lysate and/or column eluate from an affinity or cation exchange or size exclusion chromatography. Anion exchange chromatography can also be used to reduce and thereby control the amount of empty rAAV in the eluate. For example, the anion exchange column having full and empty rAAV bound thereto can be washed with a solution comprising NaCl at a modest concentration (e.g., about 100-125 mM, such as 110-115 mM) and a portion of the empty rAAV can be eluted in the flow through without substantial elution of the full rAAV. Subsequently, full rAAV bound to the anion exchange column can be eluted using a solution comprising NaCl at a higher concentration (e.g., about 130-300 mM NaCl), thereby producing a column eluate with reduced or depleted amounts of empty rAAVs and proportionally increased amounts of full rAAV comprising an rAAV vector.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, CA, USA); UNOSPHERE Q (strong anion-exchanger available from BioRad, Hercules, CA, USA); POROS 50HQ (strong anion-exchanger available from Applied Biosystems, Foster City, CA, USA); POROS XQ (strong anion-exchanger available from Applied Biosystems, Foster City, CA, USA); POROS SOD (weak anion-exchanger available from Applied Biosystems, Foster City, CA, USA); POROS 50PI (weak anion-exchanger available from Applied Biosystems, Foster City, CA, USA); Capto Q, Capto XQ, Capto Q ImpRes, and SOURCE 30Q (strong anion-exchanger available from GE healthcare, Marlborough, MA, USA); DEAE SEPHAROSE (weak anion-exchanger available from Amersham Biosciences, Piscataway, NJ, USA); Q SEPHAROSE (strong anion-exchanger available from Amersham Biosciences, Piscataway, NJ, USA). Additional exemplary anion exchange resins include aminoethyl (AE), diethylaminoethyl (DEAE), diethylaminopropyl (DEPE) and quaternary amino ethyl (QAE).

A commercial manufacturing process to purify recombinant AAV particles intended as a product to treat human disease should achieve the following objectives: 1) consistent particle purity, potency and safety; 2) manufacturing process scalability; and 3) acceptable cost of manufacturing.

Exemplary processes for recombinant AAV particle purification are reported in WO 2019/006390.

Methods to determine infectious titer of rAAV particles containing a transgene are known in the art (see, e.g., Zhen et al., Hum. Gene Ther. 15 (2004) 709). Methods for assaying for empty rAAV and full rAAV with packaged transgenes are known (see, e.g., Grimm et al., Gene Therapy 6 (1999) 1322-1330; Sommer et al., Malec. Ther. 7 (2003) 122-128).

To determine the presence or amount of degraded/denatured capsid, purified rAAV can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

### DESCRIPTION OF SPECIFIC EMBODIMENTS OF THE INVENTION

The current invention is based, at least in part, on the finding that the productivity of mammalian cells producing a recombinant adeno-associated viral particle can be increased when the cultivation is carried out at elevated pH values, such as pH 7.4-7.6.

Although the optimization of transfection parameters, DNA/reagent ratio, VCD at transfection and complexation time can improve rAAV productivity, such an increase is surpassed by far by the effect the pH value according to the current invention has.

In the following the reference value, i.e. the base value, in each table is indicated by the identifier “(100%)”.

It has been found that for the transient production of rAAV particles in HEK293 cells, the increase of the cultivation pH value from the generally used pH of 7.2 to a pH value of 7.4 or even 7.6 increases the particle yield as well as the genomic yield. As the increase of the genomic yield is higher than the increase in titer yield the full-to-empty ratio is also improved.

For example, for the transient production of rAAV2 particles in serum-free medium using an in suspension growing HEK293 cell, the increase of the cultivation pH value from the generally used pH of 7.2 to a pH value of 7.4 or even 7.6 increases the particle yield by more than 3-fold and the genomic yield by more than 10-fold.

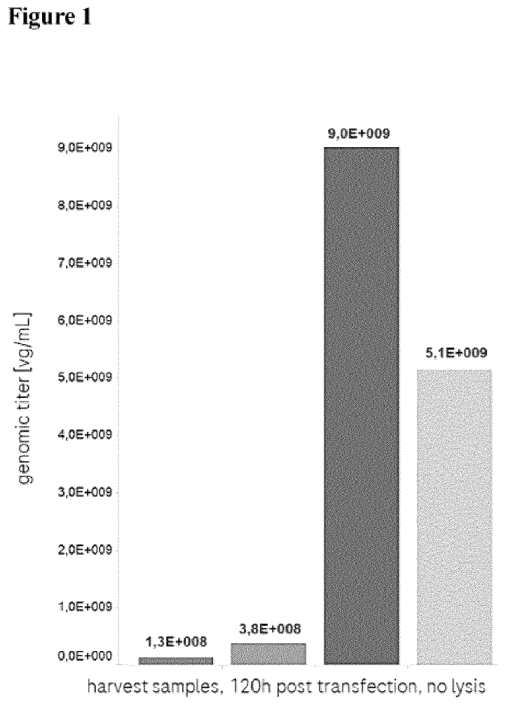

The following Tables provide exemplary data for a HEK293 cell adapted to growth in suspension in serum-free cultivation medium showing the effect of the method according to the current invention (see also FIGS. 1, 2 and 3). It can be seen that the genomic titer (vg/mL) is increased more than 10-fold if the cultivation pH value is increased from pH 7.2 to pH 7.4 or pH 7.6. Concomitantly the capsid titer (vp/mL) is increased 2- to 3-fold. Thus, as the increase in the capsid titer is lower compared to the increase in the genomic titer the full-to-empty ratio increases more than 4-fold.

| pH value | | | | | | | | |
|---|---|---|---|---|---|---|---|---|
| (harvest 72 | | | | | full/empty | full/empty | | |
| hours after | vg/mL | vg/mL | vp/mL | vp/mL | ratio | ratio | | |
| transfection) | (absolute) | (relative) | (absolute) | (relative) | (absolute) | (relative) | set | run |
| 7.0 | 4.23E+08 | 88% | 8.10E+09 | 32% | 4.96% | 263% | #1 | #1 |
| 7.2 (100%) | 4.80E+08 | 100% | 2.50E+10 | 100% | 1.89% | 100% | #1 | #2 |
| 7.4 | 8.95E+09 | 1865% | 9.60E+10 | 384% | 8.53% | 452% | #1 | #3 |
| 7.6 | 5.33E+09 | 1110% | 5.90E+10 | 236% | 8.28% | 439% | #1 | #4 |

The yield is independent of the cultivation time for pH 7.2, pH 7.4 and pH 7.6, whereas at a pH value of 7.0 the yield decreases with increasing cultivation time. This is shown in the following Table. Thus, at pH values of 7.4 and 7.6 the process is more robust and results in increased yields compared to pH 7 or pH 7.2, respectively.

| | | | | | full/empty | full/empty | | |
|---|---|---|---|---|---|---|---|---|
| | vg/mL | vg/mL | vp/mL | vp/mL | ratio | ratio | | |
| | (absolute) | (relative) | (absolute) | (relative) | (absolute) | (relative) | set | run |
| pH 7.0 | | | | | | | | |
| harvest 72 | 4.23E+08 | 100% | 8.10E+09 | 100% | 5.22% | 100% | #1 | #1 |
| hours after | | | | | | | | |
| transfection | | | | | | | | |
| (100%) | | | | | | | | |
| harvest 96 | 2.04E+08 | 48% | 5.50E+09 | 68% | 3.71% | 71% | #1 | #1 |
| hours after | | | | | | | | |
| transfection | | | | | | | | |
| harvest 120 | 1.30E+08 | 31% | 4.60E+09 | 57% | 2.83% | 54% | #1 | #1 |
| hours after | | | | | | | | |
| transfection | | | | | | | | |
| pH 7.2 | | | | | | | | |
| harvest 72 | 4.80E+08 | 100% | 2.50E+10 | 100% | 1.92% | 100% | #1 | #2 |
| hours after | | | | | | | | |
| transfection | | | | | | | | |
| (100%) | | | | | | | | |
| harvest 96 | 5.01E+08 | 104% | 2.30E+10 | 92% | 2.18% | 113% | #1 | #2 |
| hours after | | | | | | | | |
| transfection | | | | | | | | |
| harvest 120 | 3.75E+08 | 78% | 2.40E+10 | 96% | 1.56% | 81% | #1 | #2 |
| hours after | | | | | | | | |
| transfection | | | | | | | | |
| pH 7.4 | | | | | | | | |
| harvest 72 | 8.95E+09 | 100% | 9.60E+10 | 100% | 9.33% | 100% | #1 | #3 |
| hours after | | | | | | | | |
| transfection | | | | | | | | |
| (100%) | | | | | | | | |
| harvest 96 | 8.86E+09 | 99% | 9.40E+10 | 98% | 9.43% | 101% | #1 | #3 |
| hours after | | | | | | | | |
| transfection | | | | | | | | |
| harvest 120 | 9.02E+09 | 101% | 9.60E+10 | 100% | 9.39% | 101% | #1 | #3 |
| hours after | | | | | | | | |
| transfection | | | | | | | | |
| pH 7.6 | | | | | | | | |
| harvest 72 | 5.33E+09 | 100% | 5.90E+10 | 100% | 9.03% | 100% | #1 | #4 |
| hours after | | | | | | | | |
| transfection | | | | | | | | |
| (100%) | | | | | | | | |
| harvest 96 | 5.10E+09 | 96% | 5.90E+10 | 100% | 8.64% | 96% | #1 | #4 |
| hours after | | | | | | | | |
| transfection | | | | | | | | |
| harvest 120 | 5.15E+09 | 97% | 5.80E+10 | 98% | 8.87% | 98% | #1 | #4 |
| hours after | | | | | | | | |
| transfection | | | | | | | | |

The effect of the change of the pH value from pH 7.2 to pH 7.4 or pH 7.6 does by far exceed the titer increase obtained by optimizing the process conditions, such as, e.g., a change of the transfection reagent or the addition of feeding as shown in the following Table. The titer increase by a concomitant change of the cultivation pH value, the transfection reagent and the addition of a feed is shown in the third data row.

| harvest | | | | | full/empty | full/empty | | |
|---|---|---|---|---|---|---|---|---|
| 72 h post | vg/mL | vg/mL | vp/mL | vp/mL | ratio | ratio | | |
| transfection | (absolute) | (relative) | (absolute) | (relative) | (absolute) | (relative) | set | run |
| pH 7.2, | 4.80E+08 | 100% | 2.50E+10 | 100% | 1.92% | 100% | #1 | #2 |
| transfection | | | | | | | | |
| with PEI, no | | | | | | | | |
| feed (100%) | | | | | | | | |
| pH 7.2, | 9.39E+08 | 195% | 1.20E+10 | 48% | 7.83% | 407% | #3 | #5 |
| transfection with | | | | | | | | |
| FectoVIR(TM), | | | | | | | | |
| feed | | | | | | | | |
| pH 7.4, | 1.22E+10 | 2539% | 1.15E+11 | 460% | 10.62% | 553% | | |
| transfection with | | | | | | | | |
| FectoVIR(TM), | | | | | | | | |
| feed (average of | | | | | | | | |
| #3#1, #3#2, | | | | | | | | |
| #4#1, #4#2) | | | | | | | | |

For a direct comparison the titer increase resulting from a change of the cultivation pH value at the optimized transfection reagent conditions and with addition of a feed is shown in the following Table.

| | | | | | full/empty | full/empty | | |
|---|---|---|---|---|---|---|---|---|
| | vg/mL | vg/mL | vp/mL | vp/mL | ratio | ratio | | |
| | (absolute) | (relative) | (absolute) | (relative) | (absolute) | (relative) | set | run |
| pH 7.2, | 9.39E+08 | 100% | 1.20E+10 | 100% | 7.83% | 100% | #3 | #5 |
| transfection with | | | | | | | | |
| FectoVIR(TM), | | | | | | | | |
| feed (100%) | | | | | | | | |
| pH 7.4, | 1.22E+10 | 1299% | 1.15E+11 | 958% | 10.62% | 136% | | |
| transfection with | | | | | | | | |
| FectoVIR(TM), | | | | | | | | |
| feed (average of | | | | | | | | |
| #3#1, #3#2, | | | | | | | | |
| #4#1, #4#2) | | | | | | | | |

By the optimization of the cultivation conditions, the cultivation becomes less robust at a pH value of 7.2. However, at a pH value of 7.4 the process maintains its robustness. That is, by increasing the pH value from pH 7.2 to pH 7.4 the loss in process robustness due to the optimization of the reaction conditions can be counteracted. This is shown in the following Table.

| | | | | | full/empty | full/empty | | |
|---|---|---|---|---|---|---|---|---|
| | vg/mL | vg/mL | vp/mL | vp/mL | ratio | ratio | | |
| | (absolute) | (relative) | (absolute) | (relative) | (absolute) | (relative) | set | run |
| pH 7.2, | | | | | | | | |
| transfection with | | | | | | | | |
| FectoVIR(TM), feed | | | | | | | | |
| harvest 72 hours | 9.39E+08 | 100% | 1.20E+10 | 100% | 7.83% | 100% | #3 | #5 |
| post transfection | | | | | | | | |
| (100%) | | | | | | | | |
| harvest 96 hours | 5.35E+08 | 57% | 6.40E+09 | 53% | 8.36% | 107% | #3 | #5 |
| post transfection | | | | | | | | |
| pH 7.4, | | | | | | | | |
| transfection with | | | | | | | | |
| Fecto VIR(TM), feed | | | | | | | | |
| harvest 72 hours | 1.22E+10 | 100% | 1.15E+11 | 100% | 10.62% | 100% | | |
| post transfection | | | | | | | | |
| (100%) (average | | | | | | | | |
| of #3#1, #3#2, | | | | | | | | |
| #4#1, #4#2) | | | | | | | | |
| harvest 96 hours | 1.10E+10 | 90% | 9.90E+10 | 86% | 11.08% | 104% | #4 | #2 |
| post transfection | | | | | | | | |

For example, for the transient production of rAAV2 particles in serum-free medium using the commercially available HEK 293 Expi cell under the optimized conditions as described above, the particle yield can be further increased by about 1.7-fold and the genomic yield by about 1.8-fold, i.e. by 80%. The data is shown in the following Table.

| | | | | | | | | |
|---|---|---|---|---|---|---|---|---|
| pH 7.4, | | | | | full/empty | full/empty | | |
| transfection with | vg/mL | vg/mL | vp/mL | vp/mL | ratio | ratio | | |
| FectoVIR(TM), feed | (absolute) | (relative) | (absolute) | (relative) | (absolute) | (relative) | set | run |

| | | | | | | |
|---|---|---|---|---|---|---|
| serum-free | 1.22E+10 | 100% | 1.15E+11 | 100% | 10.60% | 100% |
| suspension | | | | | | |
| HEK293 | | | | | | |
| harvest 72 hours | | | | | | |
| post transfection | | | | | | |
| (100%) (average | | | | | | |
| of #3#1, #3#2, | | | | | | |
| #4#1, #4#2) | | | | | | |
| commercial | 2.21E+10 | 181% | 1.98E+11 | 172% | 11.20% | 106% |
| HEK 293 Expi | | | | | | |
| harvest 72 hours | | | | | | |
| post transfection | | | | | | |
| (average of | | | | | | |
| #3#3, #3#4, | | | | | | |
| #4#3, #4#4) | | | | | | |

The examples and figures are provided to aid the understanding of the present invention, the true scope of which is set forth in the appended claims. It is understood that modifications can be made in the procedures set forth without departing from the spirit of the invention.

### EXAMPLES

### Materials

### Cell Lines

Commercially available HEK293 cells were used for producing AAV particles using transient transfection with three plasmids.

### Cultivation Materials

Cultivation media and supplements were used according to the operating instructions of the supplier. Media and feeds were stored at 4° C. in the dark and consumed according to the manufacturer's instructions. Correction agents were stored at room temperature (glucose solution; sodium carbonate solution; defoamer solution).

### Example 1

### Cultivation of HEK293 Cells and Production of Recombinant AAV Preparations

Generally, the cultivation methods have been adapted from standard protocols (see, e.g., Lindl, T., “Zell-und Gewebekultur: Einführung in die Grundlagen sowie ausgewahlte Methoden und Anwendungen”, Spektrum Akademischer Verlag GmbH, Heidelberg/Berlin, 2002) and operating instructions of the respective supplier.

### Pre-Cultivation

HEK cells were thawed and propagated in shake flasks at 37° C., 85% humidity, a pCO2 of 5% and a shaking frequency of 120 rpm for two to three weeks in cultivation medium. Cells were split every three to four days and expanded in medium to the volume required for inoculation of the production cultivation.

### Production Cultivation

For producing recombinant AAV particles, the respective pre-cultivated HEK293 cells were cultured in the respective reactor in a batch or fed-batch process under the indicated conditions.

Set 1—rAAV Particle Preparation with Particles Comprising a Capsid Variant Derived from AAV2 Serotype and Therapeutic Transgene:
reactor: Ambr250
cell line: suspension HEK293 adapted so serum-free medium
cultivation medium: HEK ViP NB+8 mM glutamine+insulin
feed: no (batch)
temperature 37° C.
speed: ˜450 rpm
cultivation time after inoculation: 144 hours
transfection: transient; three plasmids; ratio˜1:2.5:2 (transgene:rep/cap:helper)
transfection: ˜24 hours post inoculation
transfection reagent: PEIpro™
transfection reagent: DNA ratio: ˜2:1
DNA concentration: ˜3 μg/mL
transfection VCD: ˜30 E+05 cells/mL
transfection: transfection mix in 1/3 fresh cultivation medium
lysis: no

| | | | | Genomic | Capsid | |
|---|---|---|---|---|---|---|
| | | time post | | titer | titer | Full/Empty |
| set | run | transfection | pH | [vg/mL] | [vp/mL] | [%] |
| #1 | #1 | 72 | 7 | 4.23E+08 | 8.10E+09 | 5.22 |
| #1 | #1 | 96 | 7 | 2.04E+08 | 5.50E+09 | 3.71 |
| #1 | #1 | 120 | 7 | 1.30E+08 | 4.60E+09 | 2.83 |
| #1 | #2 | 72 | 7.2 | 4.80E+08 | 2.50E+10 | 1.92 |
| #1 | #2 | 96 | 7.2 | 5.01E+08 | 2.30E+10 | 2.18 |
| #1 | #2 | 120 | 7.2 | 3.75E+08 | 2.40E+10 | 1.56 |
| #1 | #3 | 72 | 7.4 | 8.95E+09 | 9.60E+10 | 9.33 |
| #1 | #3 | 96 | 7.4 | 8.86E+09 | 9.40E+10 | 9.43 |
| #1 | #3 | 120 | 7.4 | 9.02E+09 | 9.60E+10 | 9.39 |
| #1 | #4 | 72 | 7.6 | 5.33E+09 | 5.90E+10 | 9.03 |
| #1 | #4 | 96 | 7.6 | 5.10E+09 | 5.90E+10 | 8.64 |
| #1 | #4 | 120 | 7.6 | 5.15E+09 | 5.80E+10 | 8.87 |

Set 2—rAAV Particle Preparation with Particles Comprising Capsid Variant Derived from AAV2 Serotype and Therapeutic Transgene:
reactor: Ambr250
cell line: suspension HEK293 adapted so serum-free medium
cultivation medium: HEK ViP NB+8 mM glutamine+insulin
feed: no (batch)
temperature 37° C.
speed: ˜450 rpm
cultivation time after inoculation: 120 hours
transfection: transient; three plasmids; ratio˜1:2.5:2 (transgene:rep/cap:helper)
transfection: ˜24 hours post inoculation
transfection reagent: PEIpro™
transfection reagent: DNA ratio: ˜2:1
DNA concentration: ˜3 μg/mL
transfection VCD: ˜30 E+05 cells/mL
transfection: transfection mix in ⅓ fresh cultivation medium
lysis: no

| | | | | Genomic | Capsid | |
|---|---|---|---|---|---|---|
| | | time post | | titer | titer | Full/Empty |
| set | run | transfection | pH | [vg/mL] | [vp/mL] | [%] |
| #2 | #1 | 96 | 7 | 4.00E+08 | 5.50E+09 | 7.27 |
| #2 | #2 | 96 | 7.4 | 7.70E+09 | 9.90E+10 | 7.78 |

### Set 3 and 4—DoE:

rAAV particle preparation: 1) with particles comprising capsid variant derived from AAV2 serotype and therapeutic transgene; 2) with particles comprising an AAV2 wild type capsid and green fluorescent protein (GFP) transgene:
reactor: Ambr15
cell line: 1) suspension HEK293 adapted so serum-free medium; 2) HEK293 Expi cultivation medium: HEK ViP NB+8 mM glutamine+insulin
feed: 1) no (batch); 2) medium and glucose feed post transfection temperature 37° C.
speed: ˜450 rpm
cultivation time after inoculation: 120 hours
transfection: transient; three plasmids; ratio 1)˜1:2.5:2 or 2)˜1:1:1 (transgene:rep/cap:helper)
transfection: 24 hours post inoculation
transfection reagent: 1) PEI+free PEI+valproic acid; 2) FectoVIR™-AAV
transfection reagent: DNA ratio: 1)˜2.5:1; 2)˜1.5:1
DNA concentration: 1)˜3 μg/mL; 2)˜2 μg/mL
transfection VCD: ˜30 E+05 cells/mL
transfection: transfection mix in ⅓ fresh cultivation medium
lysis: 1) no; 2) yes

| | | time | | | | | | | | Plasmid | reagent/ | Genomic | Capsid | Full/ |
|---|---|---|---|---|---|---|---|---|---|---|---|---|---|---|
| | | post | | | Cell | | | | | conc. | DNA | titer | titer | Empty |
| set | run | transfection | pH | AAV | Line | Ratio | feed | lysis | transfection | [μg/mL] | ratio | [vg/mL] | [vp/mL] | [%] |
| #3 | #1 | 72 | 7.4 | 1) | 1) | 1) | 2) | 1) | 2) | 1) | 2) | 1.18E+10 | 1.1E+11 | 10.75 |
| #3 | #1 | 96 | 7.4 | 1) | 1) | 1) | 2) | 1) | 2) | 1) | 2) | 1.05E+10 | 9.4E+10 | 11.20 |
| #3 | #2 | 72 | 7.4 | 1) | 1) | 1) | 2) | 1) | 2) | 1) | 2) | 1.24E+10 | 1.1E+11 | 11.28 |
| #3 | #2 | 96 | 7.4 | 1) | 1) | 1) | 2) | 1) | 2) | 1) | 2) | 1.19E+10 | 9.9E+10 | 12.04 |
| #3 | #3 | 72 | 7.4 | 1) | 2) | 1) | 2) | 1) | 2) | 1) | 2) | 2.07E+10 | 1.7E+11 | 12.19 |
| #3 | #3 | 96 | 7.4 | 1) | 2) | 1) | 2) | 1) | 2) | 1) | 2) | 2.06E+10 | 1.6E+11 | 12.87 |
| #3 | #4 | 72 | 7.4 | 1) | 2) | 1) | 2) | 1) | 2) | 1) | 2) | 2.29E+10 | 2.0E+11 | 11.47 |
| #3 | #4 | 96 | 7.4 | 1) | 2) | 1) | 2) | 1) | 2) | 1) | 2) | 2.52E+10 | 1.9E+11 | 13.27 |
| #3 | #5 | 72 | 7.2 | 1) | 1) | 1) | 2) | 1) | 2) | 1) | 2) | 9.39E+08 | 1.2E+10 | 7.83 |
| #3 | #5 | 96 | 7.2 | 1) | 1) | 1) | 2) | 1) | 2) | 1) | 2) | 5.35E+08 | 6.4E+09 | 8.36 |
| #3 | #6 | 72 | 7.0 | 1) | 1) | 1) | 1) | 1) | 2) | 1) | 2) | 6.29E+08 | 4.4E+09 | 14.30 |
| #3 | #6 | 96 | 7.0 | 1) | 1) | 1) | 1) | 1) | 2) | 1) | 2) | 3.46E+08 | 2.5E+09 | 13.85 |
| #3 | #7 | 72 | 7.2 | 1) | 2) | 1) | 2) | 1) | 2) | 1) | 2) | 1.39E+10 | 1.1E+11 | 12.61 |
| #3 | #7 | 96 | 7.2 | 1) | 2) | 1) | 2) | 1) | 2) | 1) | 2) | 1.60E+10 | 1.0E+11 | 16.01 |
| #3 | #8 | 72 | 7.0 | 1) | 2) | 1) | 1) | 1) | 2) | 1) | 2) | 1.94E+10 | 1.0E+11 | 19.43 |
| #3 | #8 | 96 | 7.0 | 1) | 2) | 1) | 1) | 1) | 2) | 1) | 2) | 2.01E+10 | 9.0E+10 | 22.30 |
| #3 | #9 | 72 | 7.4 | 1) | 2) | 2) | 2) | 1) | 1) | 2) | 1) | 5.66E+09 | 6.6E+10 | 8.57 |
| #3 | #9 | 72 | 7.4 | 1) | 2) | 2) | 2) | 2) | 1) | 2) | 1) | 1.43E+10 | 9.7E+10 | 14.76 |
| #3 | #10 | 72 | 7.0 | 2) | 1) | 1) | 1) | 1) | 2) | 1) | 2) | 1.88E+09 | 1.6E+10 | 11.73 |
| #3 | #10 | 96 | 7.0 | 2) | 1) | 1) | 1) | 1) | 2) | 1) | 2) | 1.67E+09 | 1.7E+10 | 9.82 |
| #3 | #11 | 72 | 7.4 | 2) | 1) | 1) | 2) | 1) | 2) | 1) | 2) | 6.81E+09 | 4.7E+10 | 14.48 |
| #3 | #11 | 96 | 7.4 | 2) | 1) | 1) | 2) | 1) | 2) | 1) | 2) | 4.43E+09 | 6.7E+10 | 6.61 |
| #3 | #12 | 72 | 7.4 | 2) | 1) | 2) | 2) | 1) | 1) | 2) | 1) | 2.29E+09 | 3.3E+10 | 6.95 |
| #3 | #12 | 72 | 7.4 | 2) | 1) | 2) | 2) | 2) | 1) | 2) | 1) | 8.27E+10 | 5.9E+11 | 14.01 |

| | | time | | | | | | | | Plasmid | reagent/ | Genomic | Capsid | Full/ |
|---|---|---|---|---|---|---|---|---|---|---|---|---|---|---|
| | | post | | | Cell | | | | | conc. | DNA | titer | titer | Empty |
| set | run | transfection | pH | AAV | Line | Ratio | feed | lysis | transfection | [μg/mL] | ratio | [vg/mL] | [vp/mL] | [%] |
| #4 | #1 | 72 | 7.4 | 1) | 1) | 1) | 2) | 1) | 2) | 1) | 2) | 1.11E+10 | 1.1E+11 | 10.13 |
| #4 | #1 | 96 | 7.4 | 1) | 1) | 1) | 2) | 1) | 2) | 1) | 2) | 1.01E+10 | 9.3E+10 | 10.82 |
| #4 | #2 | 72 | 7.4 | 1) | 1) | 1) | 2) | 1) | 2) | 1) | 2) | 1.34E+10 | 1.3E+11 | 10.32 |
| #4 | #2 | 96 | 7.4 | 1) | 1) | 1) | 2) | 1) | 2) | 1) | 2) | 1.13E+10 | 1.1E+11 | 10.26 |
| #4 | #3 | 72 | 7.4 | 1) | 2) | 1) | 2) | 1) | 2) | 1) | 2) | 2.26E+10 | 2.2E+11 | 10.28 |
| #4 | #3 | 96 | 7.4 | 1) | 2) | 1) | 2) | 1) | 2) | 1) | 2) | 2.45E+10 | 2.1E+11 | 11.65 |
| #4 | #4 | 72 | 7.4 | 1) | 2) | 1) | 2) | 1) | 2) | 1) | 2) | 2.20E+10 | 2.0E+11 | 10.98 |
| #4 | #4 | 96 | 7.4 | 1) | 2) | 1) | 2) | 1) | 2) | 1) | 2) | 2.27E+10 | 2.4E+11 | 9.44 |
| #4 | #5 | 72 | 7.4 | 1) | 1) | 2) | 2) | 1) | 1) | 2) | 1) | 2.52E+09 | 6.2E+10 | 4.07 |
| #4 | #5 | 72 | 7.4 | 1) | 1) | 2) | 2) | 2) | 1) | 2) | 1) | 4.61E+09 | 7.9E+10 | 5.84 |
| #4 | #6 | 72 | 7.0 | 2) | 2) | 2) | 1) | 1) | 1) | 2) | 1) | 1.14E+08 | 6.2E+08 | 18.45 |
| #4 | #6 | 72 | 7.0 | 2) | 2) | 2) | 1) | 2) | 1) | 2) | 1) | 1.52E+09 | 1.9E+10 | 7.98 |
| #4 | #7 | 72 | 7.0 | 2) | 2) | 1) | 1) | 1) | 2) | 1) | 2) | 1.25E+09 | 1.2E+10 | 10.45 |
| #4 | #7 | 96 | 7.0 | 2) | 2) | 1) | 1) | 1) | 2) | 1) | 2) | 5.48E+08 | 7.6E+09 | 7.21 |
| #4 | #8 | 72 | 7.0 | 1) | 1) | 2) | 1) | 1) | 1) | 2) | 1) | 7.44E+08 | 7.6E+09 | 9.79 |
| #4 | #8 | 72 | 7.0 | 1) | 1) | 2) | 1) | 2) | 1) | 2) | 1) | 2.84E+09 | 5.0E+10 | 5.68 |
| #4 | #9 | 72 | 7.4 | 2) | 2) | 1) | 2) | 1) | 2) | 1) | 2) | 4.35E+09 | 5.0E+10 | 8.70 |
| #4 | #9 | 96 | 7.4 | 2) | 2) | 1) | 2) | 1) | 2) | 1) | 2) | 6.60E+09 | 7.1E+10 | 9.30 |
| #4 | #10 | 72 | 7.0 | 2) | 1) | 2) | 1) | 1) | 1) | 2) | 1) | 1.10E+09 | 1.4E+10 | 7.86 |
| #4 | #10 | 72 | 7.0 | 2) | 1) | 2) | 1) | 2) | 1) | 2) | 1) | 4.31E+09 | 7.7E+10 | 5.60 |
| #4 | #11 | 72 | 7.0 | 1) | 2) | 2) | 1) | 1) | 1) | 2) | 1) | not | 4.3E+09 | not |
| | | | | | | | | | | | | determined | | determined |
| #4 | #11 | 72 | 7.0 | 1) | 2) | 2) | 1) | 2) | 1) | 2) | 1) | not | 4.6E+10 | not |
| | | | | | | | | | | | | determined | | determined |
| #4 | #12 | 72 | 7.4 | 2) | 2) | 2) | 2) | 1) | 1) | 2) | 1) | 4.26E+09 | 2.0E+10 | 21.31 |
| #4 | #12 | 72 | 7.4 | 2) | 2) | 2) | 2) | 2) | 1) | 2) | 1) | 6.86E+09 | 8.2E+10 | 8.36 |

### Example 2

### Lysis

In case lysis was included in the process, it has been performed as follows: To release the AAV particles into the cell culture broth, 5% (v/v) of lysis buffer (10% Triton CG 110, 40 mM MgCl₂) was added to the culture broth. Additionally, 100 U/ml Benzonase™ nuclease (Merck). The cell culture broth was then incubated for about one hour at 37° C. with stirring, without aeration and pH control. After the respective incubation, 5 M NaCl solution was added and the lysate was sterile filtered.

### Example 3

### AAV Particle Purification

For the affinity chromatography step a column comprising 10.5 mL AAVX resin from Thermo Fisher was used on an Akta Avant 25 chromatography system. The system was run at a flow rate of about 300 cm/h. After equilibration with buffer A (1×PBS, pH 7.4, 0.001% Pluronic F-68) 200 mL of the lysed culture broth was applied to the column followed by 2 wash steps with equilibration buffer and 0.5 M NaCl, pH 6.0, respectively. AAV particles were eluted with 0.1 M sodium citrate solution, pH 2.4. The pH of the eluate was adjusted to pH 7.5 by addition of 2 M Tris, pH 10.

| step | buffer | column volumes [CV] |
|---|---|---|
| equilibration | 1 × PBS, pH 7.4 | 3 |
| load | lysate | |
| wash I | 1 × PBS, pH 7.4 | 4 |
| wash II | 0.5M sodium chloride pH 6.0 | 4 |
| wash III | 1 × PBS, pH 7.4 | 4 |
| elution | 0.1M sodium citrate pH 2.4 | 3 |

### Example 4

### Analytical Methods

Enzyme-Linked Immunosorbent Assay (ELISA) for Total Titer Determination For AAV capsid titer determination the kit from PROGEN (Cat. no PRAAV8) was used according to the manufacturer's instructions.

In short, this assay is a sandwich ELISA using as capture antibody a recombinant AAV capsid specific antibody and a biotin-labeled detection antibody.

The wells of the pre-coated multi-titer plate (MTP) were incubated overnight with 100 μL standard, sample or control, respectively, at 4° C. The next day the wells were washed three times with ASSB buffer (lx) as provided in the kit. Thereafter 100 μL per well of a solution comprising the biotinylated detection antibody (diluted according to the manufacturer's instructions) were added and incubated for two hours at room temperature with shaking. Afterwards, the wells were washed three times with ASSB buffer (lx) as provided in the kit. In the next step 100 μl of a solution comprising horseradish peroxidase conjugated to streptavidin was added to each well and incubated for 30 min. At room temperature with shaking. Afterwards, the wells were washed three times with ASSB buffer (1×) as provided in the kit. For color reaction 100 μL of a solution comprising ABTS prepared according to the manufacturer's instructions was added to each well and incubated with shaking. The color intensity was determined suing an MTP-ELISA-Reader Versa Max (Molecular Devices) at 405 nm with a reference wavelength of 490 nm until the difference in the extinction between blank and the standard with the highest concentration reaches about 1.5.

Each sample, standard and control was measured in duplicate.

The amount of capsids (capsids/mL) was calculated based on a standard curve determined by a 4-parameter fitting, e.g. according to the WiemerRodbard algorithm, using the average values of the standards.

Digital Droplet Polymerase Chain Reaction (ddPCR) for Genomic Titer Determination

### Reagents for Enzymatic Sample Treatment:

1) DNase I buffer (NEB): 100 mM Tris-HCl, pH 7.6, 25 mM MgSO₄, 5 mM CaCl₂)
2) DNase I (NEB): 0.2 U/μL
3) Proteinase K (NEB; approx. 20 mg/mL=800 U/mL): 16 U/mL
4) proteinase K buffer (BioRad): 400 mM Tris-HCl, 20 mM EDTA, 2000 mM NaCl, 1% SDS, pH 8
5) sodium dodecyl sulfate (SDS) solution: 10% (w/v)

### Enzymatic Sample Treatment:

mix 30 μL H2O, 5 μL DNase I buffer, 5 μL DNase I, 10 μL sample
incubate at 37° C. for 30 min.
heat to 75° C. for 15 min. to obtain an incubated DNase I-Mix
short cool down and centrifugation
mix 42 μL H2O+2 μL proteinase K+5 μL proteinase K buffer+1 μL 10% SDS solution and add the incubated DNase I-Mix
incubate for 60 min. at 50° C.
heat to 95° C. for 15 min.
cooling to 4° C.
   ddPCR:

For viral genome titration, a duplexing ddPCR assay was performed. Primer and probes were designed against the used CMV promoter and against the polyA/3′UTR sequence. The PCR mastermix was prepared according to the following Table (droplet digital PCR guide—Bio-Rad).

| components | volume per well [μL] | final concentration |
|---|---|---|
| Supermix (2x) | 11 | 1x |
| 20 μM CMV primer fwd. | 0.99 | 900 nM |
| 20 μM CMV primer rev | 0.99 | 900 nM |
| 20 μM CMV probe | 0.275 | 250 nM |
| template | 5.5 | — |
| water | 0.99 | — |
| total | 22 | |

The prepared mastermix was pipetted into a 96 well plate with 16.5 μL per well. Then, dilution series of the pretreated samples were conducted: 10 μL of samples were transferred with LoRentention Tips into 90 μL water in LoBind Tubes and thoroughly mixed. Thereafter, 5.5 μL of the samples were added to the mastermix solution in the 96 well plate in several dilution steps. The plate was sealed at 180° C., vortexed at 2,200 rpm for 1 min. and centrifuged at 1,000 rpm for another 1 min. With an automatic droplet generator device, which takes 20 μL PCR mix out of each well, up 20,000 droplets per well were produced and transferred into another 96 well plate. After sealing the droplet plate at 180° C., a PCR run was carried out. The respective conditions are shown in the following Table.

| number of | | | final | |
|---|---|---|---|---|
| cycles | denaturation | annealing | elongation | end |
| 1 | 94° C., 10 min. | | | |
| 39 | 94° C., 30 sec. | 58° C., 1 min. | | |
| 1 | | | 98° C., | constant at |
| | | | 10 min. | 12° C. |

In a droplet reader, the fluorescence signal was measured for each droplet. The QuantaSoft software processed the reader data and calculated copy numbers per 20 μL well for the target sequences. Initial sample titers can be determined with following equation:

$$copy number [ \frac{copies}{mL} ] = \frac{output [ \frac{copies}{20 µL well} ]}{5 [ \frac{µL sample}{20 µL well} ]} \cdot dilution factor \cdot 1000 [ \frac{µL}{mL} ]$$

1. A method for producing a recombinant adeno-associated viral particle preparation (rAAVp) comprising the step of cultivating a HEK293 cell comprising expression cassettes for a non-adeno-associated viral gene, which is interspaced between two AAV inverted terminal repeats (ITRs), an adeno-associated virus rep gene, an adeno-associated virus cap gene, an adeno-associated virus E1A gene, an adeno-associated virus E1B gene, an adeno-associated virus E2A gene, an adeno-associated virus E4orf6 and optionally an adeno-associated virus VA RNA gene, and thereby producing the rAAVp,
wherein the cultivating is at a pH value in the range of and including pH 7.4 to pH 7.6.

2. The method according to claim 1, wherein the yield of the rAAVp produced by the cultivating at a pH value in the range of and including pH 7.4 to pH 7.6 is higher than the yield of a rAAVp produced by a cultivating at a pH value in the range of and including pH 7.0 to pH 7.2.

3. The method according to claim 2, wherein the rAAVp produced by the cultivating at a pH value in the range of and including pH 7.4 to pH 7.6 has a higher percentage of full particles than a rAAVp produced by a cultivating at a pH value in the range of and including pH 7.0 to pH 7.2.

4. The method according to claim 1, wherein the rAAVp is a therapeutic rAAVp.

5. The method according to claim 1, wherein the rAAVp comprises recombinant adeno-associated viral particles (rAAVs) comprising at least one coding nucleic acid sequence interspaced between two adeno-associated viral inverted terminal repeats.

6. The method according to claim 1, wherein the rAAV is of the serotype AAV2 or a variant thereof.

7. The method according to claim 1 wherein the cultivating encompasses the inoculation of the bioreactor and the harvest of the rAAVp.

8. The method according to claim 1, wherein one or more or all of the expression cassettes for the non-adeno-associated viral gene, which is interspaced between two AAV ITRs, for the adeno-associated virus rep gene, for the adeno-associated virus cap gene, for the adeno-associated virus E2A gene, for the adeno-associated virus E4orf6 and optionally for the adeno-associated virus VA RNA gene are introduced into the mammalian cell after the inoculation of the bioreactor.

9. The method according to claim 1, wherein the method further comprises after the cultivating step the step of isolating the rAAV from the cells and/or the cultivation medium and optionally purifying the rAAV.

10. The method according to claim 9, wherein the purifying is by a sequence of chromatography steps wherein the first is an affinity chromatography, followed by an anion exchange chromatography or a cation exchange chromatography, and an optional size exclusion chromatography.

11. A pharmaceutical composition comprising the rAAVp obtained with a method according to claim 1.

12. A pharmaceutical composition comprising the rAAVp obtained with a method according to claim 1 and a pharmaceutically acceptable excipient.

13. Use of the method according to claim 1 for increasing the yield of a recombinantly produced rAAVp.

14. Use of the method according to claim 1 for increasing the percentage of full particles in a rAAVp.
